(19) United States
(12) Patent Application Publication
(10) Pub. No.: US 2009/0117102 A1
(43) Pub. Date: May 7, 2009

(54) Methods and compositions using CD3 agonists

(76) Inventor: Antonio Cruz, Ontario (CA)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/630,946

(22) PCT Filed: Jun. 29, 2005
(86) PCT No.: PCT/CA05/01024

§ 371 Date: Feb. 11, 2008

Related U.S. Application Data
(60) Provisional application No. 60/584,635, filed on Jul. 1, 2004.

(51) Int. Cl. A61K 39/395; A61K 38/10; A61K 38/08; A61K 35/12; A61P 3/10

(52) U.S. Cl. 424/133.1; 424/173.1; 514/12; 514/13; 514/14; 514/17; 424/93.7

(57) ABSTRACT

Compositions of CD3 antibody and gastrin and uses thereof in the prevention and intervention of diabetes.

### FIELD OF THE INVENTION

The invention relates generally to compositions, conjugates, and methods comprising a CD3 agonist, and uses thereof.

### BACKGROUND OF THE INVENTION

There are estimated to be 18 million people in the United States, or 6.3% of the population, who have diabetes. Diabetes results from an absence or relative deficiency of insulin production or utilization. Type 1 diabetes is characterized by insufficient production of insulin and Type 2 diabetes is characterized by insufficient utilization of insulin.

Type 1 diabetes results from cellular autoimmune destruction of the pancreatic beta cells in the islets of Langerhans of the pancreas, leading to an absolute insulin deficiency. Autoimmunity is also a factor in Latent Autoimmune Diabetes in the Adult (LADA) which is clinically similar to Type 2 diabetes but which is positive for pancreatic autoantibodies. In LADA patients, autoimmunity mediated by T cells accelerates the disease process resulting in rapid progression to insulin injections in these patients. Modulation of T cells or the immune systems' response to T cells has been suggested as a means to influence the development of Type 1 diabetes and disease progression in LADA patients.

The CD3 antigen is present on mature human T cells, thymocytes and a subset of natural killer cells. It, is associated with the T cell receptor (TCR) and is responsible for the signal transduction of the TCR. Owing to its central role in modulating T cell activity, the TCR/CD3 complex has been the subject of much research aimed at developing molecules capable of binding TCR/CD3. Much of this work has focused on the development of antibodies specific for the human CD3 antigen. One such antibody is the murine monoclonal antibody OKT3 which was the first monoclonal antibody approved by the FDA. OKT3 is reported to be a potent T cell mitogen (Van Wauve, J. Immunol. 124 (1980), 2708-18; U.S. Pat. No. 4,361,539) and a potent T cell killer (Wong Transplantation 50 (1990), 683-9). Other antibodies specific for the CD3 antigen have also been reported (see Published PCT WO04106380; Published US Application No. US20040202657; U.S. Pat. No. 6,750,325; U.S. Pat. No. 6,706,265; GB No. 2249310A; and Clark et al., European J. Immunol. 1989, 19:381-388; U.S. Pat. No. 5,968,509).

The citation of any reference herein is not an admission that such reference is available as prior art to the instant invention.

### SUMMARY OF THE INVENTION

The invention provides compositions, conjugates (e.g. chimeric polypeptides), nucleic acids, and methods (e.g. combination therapies) comprising or encoding one or more CD3 agonist and one or more gastrin compound that provides beneficial effects in the treatment of conditions or diseases for which a CD3 agonist and/or gastrin compound have a therapeutic effect, including diabetes, hypertension, chronic heart failure, fluid retentive states, obesity, metabolic syndrome, and related diseases and disorders. Combinations of a CD3 agonist and a gastrin compound may be selected to provide unexpectedly additive effects or greater than additive effects i.e. synergistic effects.

A composition, conjugate, nucleic acid, or method (e.g. combination therapy) comprising or encoding a CD3 agonist and a gastrin compound employing different mechanisms to achieve maximum therapeutic efficacy, may improve tolerance to the therapy with a reduced risk of side effects that may result from higher doses or longer term monotherapies (i.e. therapies with each compound alone). A treatment of the invention can permit the use of lower doses of each compound with reduced adverse toxic effects of each compound. A suboptimal dosage may provide an increased margin of safety, and may also reduce the cost of a drug necessary to achieve prophylaxis and therapy. In addition, a treatment utilizing a single combination dosage unit may provide increased convenience and may result in enhanced compliance. Other advantages of a composition, conjugate, or combination therapy may include higher stability towards degradation and metabolism, longer duration of action, and/or longer duration of action or effectiveness at particularly low doses.

More particularly, a composition, conjugate, nucleic acid or method (e.g. combination therapy) comprising or encoding a CD3 agonist and a gastrin compound may avoid destruction of beta cells, and/or lead to an increase of beta cell function as measured by C-peptide after treatment has been discontinued. A treatment can be sustained over several years thereby having a major beneficial impact on the severity of the disease and its complications.

Broadly stated, the invention relates to compositions, conjugates, nucleic acids, and methods for the prevention, intervention, and/or treatment of a condition and/or disease comprising a therapeutically effective amount of a CD3 agonist and a gastrin compound or nucleic acid encoding same that provide beneficial effects.

The invention also contemplates a composition, preferably a pharmaceutical composition, comprising a CD3 agonist and a gastrin compound. A pharmaceutical composition may optionally comprise a pharmaceutically acceptable carrier, excipient, or vehicle.

The invention also contemplates a pharmaceutical composition, comprising one or more CD3 agonist and one or more gastrin compound that provides beneficial effects relative to each compound alone. The beneficial effects provided by a composition of the invention can include increased absorption, distribution, metabolism and/or elimination of a CD3 agonist. A composition can have increased bioavailability (absorbed more rapidly and to a higher degree) or provide enhanced therapeutic effects.

The invention also provides a pharmaceutical composition for the treatment of a condition and/or disease comprising a therapeutically effective amount of a CD3 agonist and a gastrin compound to provide a sustained beneficial effect following treatment in a pharmaceutically acceptable carrier, excipient, or vehicle. In an embodiment, the composition is in a form such that administration to a subject results in blood glucose levels that are about normal that persist in the subject for a sustained period of time after cessation of treatment. In another embodiment, the composition is in a form such that administration to a subject leads to an increase in beta cell function, in particular an increase that persists for a sustained period of time following treatment.

The invention relates to compositions, conjugates, nucleic acid constructs, and methods for proliferation and/or differentiation of islet precursor cells, mature insulin secreting cells or pancreatic insulin secreting β cells, and uses of same for preventing and/or treating a condition and/or disease described herein.

In an aspect, the invention features a composition comprising one or more CD3 agonist and optionally one or more gastrin compound in dosages effective for inducing proliferation and/or differentiation of islet precursor cells into an increased amount of islet precursor cells or mature insulin secreting cells, in particular for a sustained period following administration of the CD3 agonist and optionally gastrin compound. Proliferation of islet precursor cells may be induced ex vivo or in vivo. The composition can be in a dosage effective for inducing differentiation of an islet precursor cell into a mature insulin secreting cell. The composition can be in a pharmaceutically acceptable carrier, excipient, or vehicle.

In another aspect the invention provides a combination of a CD3 agonist and a gastrin compound that provides beneficial effects in the treatment of conditions for which either a CD3 agonist or a gastrin compound have been demonstrated to have a therapeutic effect, including but not limited to diabetes, hypertension, chronic heart failure, fluid retentive states, metabolic syndrome, obesity, and related diseases and disorders. Combinations comprising a CD3 agonist and a gastrin compound may be selected to provide unexpectedly additive effects or greater than additive effects i.e. synergistic effects.

A CD3 agonist and a gastrin compound in a composition or combination of the invention may be in a ratio selected to augment the activity of the CD3 agonist and/or gastrin compound to provide a beneficial effect.

The invention also provides a pharmaceutical composition in separate containers and intended for simultaneous or sequential administration to provide beneficial effects, comprising a CD3 agonist and a gastrin compound, both optionally together with pharmaceutically acceptable carriers, excipients, or vehicles.

The invention provides a conjugate comprising a CD3 agonist interacting with or linked to a gastrin compound. A conjugate can provide the beneficial effects described herein. In an aspect, a chimeric polypeptide is provided comprising a CD3 agonist and a gastrin compound.

The invention also provides methods for preparing compositions and conjugates of the invention that result in compositions and conjugates with beneficial effects.

In an aspect, the invention provides a method of preparing a stable pharmaceutical composition comprising one or more CD3 agonist adapted to provide beneficial effects, preferably sustained beneficial effects following treatment. A method can comprise mixing one or more CD3 agonist, and a pharmaceutically acceptable carrier, excipient, or vehicle, in particular, a pharmaceutically acceptable carrier, excipient, or vehicle effective to physically stabilize the CD3 agonist(s). After compositions have been prepared, they can be placed in an appropriate container and labeled for treatment of an indicated condition. For administration of a composition of the invention, such labeling would include amount, frequency, and method of administration. In another aspect the invention provides a method of preparing a stable pharmaceutical composition of a CD3 agonist and a gastrin compound adapted to provide beneficial effects following treatment, comprising preparing a composition comprising the CD3 agonist, a gastrin compound, and a pharmaceutically acceptable carrier, excipient, or vehicle effective to physically stabilize the CD3 agonist and gastrin compound.

In an aspect, the invention provides a nucleic acid construct comprising a nucleic acid sequence encoding a mammalian CD3 agonist operably linked to a regulatory element (e.g. a heterologous promoter) and a nucleic acid sequence encoding a mammalian gastrin compound operably linked to a regulatory element (e.g. heterologous promoter). A nucleic acid construct may additionally comprise a sequence encoding an exogenous polypeptide.

A nucleic acid molecule may be inserted into an appropriate expression vector i.e. a vector that contains the necessary regulatory elements for the transcription and translation of the inserted coding sequences. The invention provides host cells comprising or transformed with a construct or vector of the invention.

The invention provides a method for genetically modifying stem cells, islet precursor cells, mature insulin secreting cells or pancreatic insulin secreting β cells with a nucleic acid construct of the invention comprising obtaining islet precursor cells, mature insulin secreting cells or pancreatic insulin secreting c cells to be genetically modified, providing the cells ex vivo with conditions for cell proliferation, and genetically modifying the cells with the nucleic acid construct. The invention contemplates modified islet precursor cells, mature insulin secreting cells, pancreatic insulin secreting β cells or modified stem cells produced by methods of the invention. A modified cell of the invention may comprise an inducible regulatory element which when activated results in expression of the nucleic acid construct thereby effecting proliferation and/or expansion of the cells.

The invention further relates to a cell culture that comprises transformed host cells of the invention. The invention also relates to a preparation comprising modified cells or expanded or differentiated cells produced by a method of the invention. Cell preparations comprising expanded or differentiated cells can be used in a variety of methods (e.g. transplantation or grafting) and they have numerous uses in the field of medicine.

The invention further provides a method for differentiating stem cells or progenitor cells into insulin secreting cells comprising contacting the stem cells or progenitor cells with a CD3 agonist and optionally a gastrin compound or a composition or conjugate of the invention in sufficient amounts to differentiate stem cells or progenitor cells. The amount of differentiation may be significantly different compared with that achieved in the absence of the compounds, composition or conjugate. In an embodiment, the stem cells or progenitor cells are contacted with the compound(s), composition, or conjugate in culture. In another embodiment, the stem cells or progenitor cells are contacted with the compound(s), composition, or conjugate in a subject. The compound(s), composition or conjugate may be administered to a subject before, during, or after implantation of stem cells in the subject to expand and differentiate the stem cells in the subject.

The invention also relates to a method for enhancing proliferation of islet cells or insulin secreting cells in culture comprising contacting the cells with a CD3 agonist and optionally a gastrin compound, nucleic acid construct, or a composition or conjugate of the invention in sufficient amounts to enhance proliferation of the cells. The amount of proliferation may be significantly different compared with that achieved in the absence of the compounds, construct, composition or conjugate.

The invention also relates to a method for sustaining islet cells or precursor cells in culture comprising culturing the cells in the presence of a CD3 agonist and optionally a gastrin compound or a composition, nucleic acid construct, or conjugate of the invention in an amount sufficient to sustain the cells in culture. The cells may be sustained in culture for a significantly longer period of time compared with cells cultured in the absence of the compounds, constructs, composition or conjugate. Culturing cells in the presence of a CD3 agonist and optionally a gastrin compound, construct, or a composition or conjugate of the invention will be particularly useful in preparing and maintaining cells intended for transplantation.

The invention also contemplates the use of a CD3 agonist and a gastrin compound, composition, conjugate, or nucleic acid of the invention, or combination treatment of the invention for preventing, and/or ameliorating disease severity, disease symptoms, and/or periodicity of recurrence of a condition and/or disease described herein. The invention also relates to the prevention and treatment, in a subject, of conditions and/or diseases using a CD3 agonist and a gastrin compound, nucleic acid construct, composition, combination treatment, and/or conjugate of the invention. In particular, the invention provides a method for treating and/or preventing a condition and/or disease in a subject comprising administering to the subject a therapeutically effective amount of one or more CD3 agonist and one or more gastrin compound to provide beneficial effects. In an aspect the invention provides a treatment which results in sustained beneficial effects following treatment.

In an aspect, the invention provides a method for the prevention and/or intervention of a condition and/or disease discussed herein in a subject comprising administration of at least one CD3 agonist and at least one gastrin compound, or a composition or conjugate of the invention to the subject. A CD3 agonist and a gastrin compound, composition or conjugate may be directly administered to a subject or contacted with cells (e.g. stem cells or progenitor cells) and administered to a subject.

In other aspects, the invention provides a method for the prevention and/or intervention of a condition and/or disease discussed herein in a subject comprising administration of at least one CD3 agonist and at least one gastrin compound to a subject in need thereof to provide beneficial effects.

In another aspect, the invention provides a method for the prevention and/or intervention of a condition and/or disease discussed herein in a subject comprising co-administering at least one CD3 agonist and at least one gastrin compound to a subject in need thereof.

The invention has particular applications in preventing and/or treating diabetes and complications thereof. Thus, the invention relates to a method of treatment comprising administering a therapeutically effective amount of one or more CD3 agonist and a gastrin compound which upon administration to a subject with symptoms of diabetes produces beneficial effects, preferably sustained beneficial effects. In an embodiment, sustained beneficial effects are evidenced by one or more of the following: (a) an increase in C-peptide production, (b) an increase in pancreatic insulin production, (c) an increase in beta cell function; and/or (d) about normal blood glucose levels.

In an embodiment, the invention provides a method for preventing and/or treating Type 1 or Type 2 diabetes comprising administering a therapeutically effective amount of a CD3 agonist and a gastrin compound, or a composition or conjugate of the invention. In another embodiment, the invention provides a method for ameliorating progression of a condition and/or disease or obtaining a less severe stage of a condition and/or disease in a person suffering from Type 1 or Type 2 diabetes comprising administering a therapeutically effective amount of a CD3 agonist and a gastrin compound, or a composition or conjugate of the invention. The invention relates to a method of delaying the progression of impaired glucose tolerance or non-insulin requiring Type 2 diabetes to insulin requiring Type 2 diabetes comprising administering a therapeutically effective amount of a CD3 agonist and a gastrin compound, or a composition or conjugate of the invention. The invention also relates to a method of increasing the insulin synthesis capability of a subject comprising administering a therapeutically effective amount of a CD3 agonist and a gastrin compound, or a composition or conjugate of the invention.

In embodiments of methods of the invention the subject is not treated with insulin. In other embodiments of the methods of the invention the subject is treated with insulin.

The invention provides methods for treating cells using a CD3 agonist and gastrin compound of the invention, composition, conjugate or nucleic acid construct of the invention. In particular, the invention relates to a method for expanding and differentiating stem cells or progenitor cells into insulin secreting cells, enhancing proliferation of insulin secreting cells, and/or sustaining islet cells or precursor cells. Cells may be contacted with a CD3 agonist and optionally a gastrin compound in culture or in a subject.

The invention provides a method of treating a condition and/or disease comprising administering at least one CD3 agonist and optionally at least one gastrin compound, nucleic acid construct, a composition, combination treatment or conjugate of the invention, with a plurality of cells, to a subject in need thereof to thereby produce beneficial effects. In an embodiment, the compounds, nucleic acid construct, composition, and/or conjugate are administered systemically.

The invention further relates to a method for treating a subject with a condition and/or disease described herein comprising contacting ex vivo a plurality of cells with a CD3 agonist and optionally a gastrin compound, nucleic acid construct, or a composition or conjugate of the invention, optionally culturing the cells, and administering the cells to the subject in need thereof.

Methods and compositions are provided for treating diabetes in a patient in need thereof by implanting into a diabetic patient pancreatic islet cells that have been exposed in culture to a sufficient amount of a CD3 agonist and optionally a gastrin compound to increase the number of pancreatic beta cells in the islets; optionally the population of pancreatic beta cells can be grown in culture for a time sufficient to expand the population of β-cells prior to transplantation.

The invention still further relates to inducing islet neogenesis in a subject comprising contacting islet precursor cells with a CD3 agonist and optionally a gastrin compound, nucleic acid construct, composition, or conjugate of the invention in a sufficient amount to increase proliferation of islet precursor cells in the subject thereby inducing islet neogenesis.

An embodiment of the invention provides a method for preventing and/or treating diabetes, the method comprising administering to a mammal in need thereof a composition comprising a combination of a CD3 agonist and a gastrin compound in an amount sufficient to increase proliferation of islet precursor cells in pancreatic tissue, thereby preventing and/or treating the diabetes.

The invention provides methods for treating diabetes mellitus in a patient in need thereof by administering a composition comprising a CD3 agonist and optionally a gastrin compound, composition, or conjugate of the invention in an amount sufficient to effect differentiation of the patient's pancreatic islet precursor cells to mature insulin-secreting cells and/or to stimulate insulin synthesis in existing islet cells. The composition can be administered systemically or expressed in situ by host cells containing a nucleic acid construct in an expression vector wherein the nucleic acid construct comprises a coding sequence for a CD3 agonist and optionally a coding sequence for a gastrin compound, together with transcriptional and translational regulatory elements functional in pancreatic islet precursor cells.

The invention in an aspect provides a method for preventing and/or treating diabetes, the method comprising administering to a mammal in need thereof a composition comprising a CD3 agonist and a gastrin compound, composition or conjugate of the invention in an amount sufficient to increase the number of pancreatic insulin secreting β cells in the mammal, thereby preventing and/or treating the diabetes. The composition is administered systemically. The mammal is a diabetic mammal, for example, the mammal has been diabetic for an extent of 1% of the lifespan of the mammal. In general, the amount of CD3 agonist in the composition is generally substantially lower than the minimum effective dose of CD3 agonist required to reduce blood glucose or increase beta cell function in the diabetic mammal in the absence of a gastrin compound. The CD3 agonist and the gastrin compound are provided in an amount sufficient to induce differentiation of the pancreatic islet precursor cells into glucose responsive insulin secreting islet cells.

In another aspect, the invention provides a method for preventing and/or treating diabetes, the method comprising administering to a mammal in need thereof a composition comprising a combination of a CD3 agonist and optionally a gastrin compound in an amount sufficient to increase the number of pancreatic insulin secreting β cells in the mammal; and determining the amount of islet neogenesis, thereby preventing and/or treating the diabetes.

The invention features a method of enhancing islet neogenesis in a subject comprising administering a CD3 agonist and optionally a gastrin compound, a composition, or conjugate of the invention after a sufficient period of time following transplantation in the subject of islet precursor or mature insulin-secreting cells to provide sustained beneficial effects.

In an aspect, the invention provides a method for inducing pancreatic islet neogenesis in a mammal, the method comprising administering to the mammal a composition comprising a combination of a CD3 agonist and optionally a gastrin compound, in an amount sufficient to increase proliferation of islet precursor cells in pancreatic tissue, thereby inducing pancreatic islet neogenesis. The plurality of cells can be multicellular. The plurality of cells can be delivered systemically to the mammal.

In another aspect, the invention provides a method for inducing pancreatic islet neogenesis in a mammal, the method comprising administering a composition comprising a combination of a CD3 agonist and a gastrin compound, in an amount sufficient to increase the number of pancreatic insulin secreting β cells in the mammal.

In a further aspect, the invention provides a method for inducing islet neogenesis therapy in a cell of an animal, comprising contacting the cell with a nucleic acid sequence encoding a CD3 agonist and optionally a nucleic acid sequence encoding a gastrin compound operably linked to a regulatory element (e.g. an insulin promoter receptor ligand). For example, the cell is a germ cell, or the cell is an autologous cell cultured ex vivo.

Another embodiment of the invention provides a method for preventing and/or treating diabetes, the method comprising: contacting ex vivo a plurality of cells with a composition comprising a CD3 agonist and optionally a gastrin compound in an amount sufficient to increase proliferation of islet precursor cells and the amount of insulin secreting islet cells; and administering the contacted plurality of cells to a mammal in need thereof, thereby preventing and/or treating the diabetes. The cells can be autologous. The composition is provided in an amount sufficient to effect differentiation of stem cells, for example, to effect differentiation of pancreatic islet precursor cells in pancreatic tissue into mature insulin secreting islet cells. The composition is provided in an amount sufficient to increase proliferation of pancreatic islet stem cells, for example, of pancreatic islet precursor cells. Stem cells can be obtained either from a pancreatic tissue or from a non-pancreatic tissue, such as liver or bone marrow.

In another aspect, an embodiment of the invention provides a method for differentiating and optionally expanding stem cells in a diabetic recipient of the cells into insulin secreting cells, the method comprising implanting the cells in the recipient, and administering a composition containing an effective dose of a CD3 agonist and optionally a gastrin compound, or a composition or conjugate of the invention. For example, the implanted cells are obtained from a human, for example, are obtained from human pancreatic islets, human liver, human bone marrow, human umbilical cord, or human embryos. Implanting the cells into the recipient may be by a route such as injecting directly into an organ, for example, into the pancreas, the kidney, or the liver. Alternatively, the cells may be implanted by intravenous injection, for example, into the portal vein or into the hepatic vein. In certain embodiments, prior to implanting, the cells are treated ex vivo with a composition comprising a CD3 agonist, and optionally a gastrin compound or nucleic acid construct or conjugate of the invention.

The invention also contemplates the use of a composition comprising at least one CD3 agonist and optionally at least one gastrin compound for the preparation of a medicament for preventing and/or treating a condition or disease. In an embodiment, the invention relates to the use of synergistically effective amounts of at least one CD3 agonist, and at least one gastrin compound for the preparation of a medicament for preventing and/or treating a condition or disease. The invention additionally provides uses of a pharmaceutical composition and a conjugate of the invention in the preparation of medicaments for the prevention and/or treatment of diseases and conditions. The medicaments provide beneficial effects, preferably sustained beneficial effects following treatment.

In aspects of the invention, the condition and/or disease are Type 1 diabetes or LADA.

In embodiments of the invention, the CD3 agonist is an antibody reactive with CD3 or an F(ab′)₂ fragment of the antibody.

In other embodiments of the invention, the CD3 agonist is an anti-CD3 antibody selected from the group consisting of OKT3, hOKT3γl (Ala-Ala), 145 2Cl 1, YTH 12.5, YTH 12.5.14.2, or CAMPATH-3, or an F(ab′)₂ fragment of the antibody.

In particular embodiments of the invention, the CD3 agonist is anti-CD3 mAB hOKT3γl (Ala-Ala).

In particular embodiments of the invention, the CD3 agonist is anti-CD3 mAb 145 2Cl 1 or an F(ab′)₂ fragment thereof.

In embodiments of the invention, the gastrin compound is a gastrin 34 or gastrin 17.

In embodiments of the invention, the gastrin compound is a gastrin 34 or gastrin 17 where there is a methionine or a leucine at position 15, as shown in SEQ ID NOs: 6-9 herein.

A composition, conjugate, nucleic acid, or method of the invention may further comprise or encode a GLP-1 agonist. In embodiments of the invention, the GLP-1 agonist is a stable GLP-1 analog/derivative. In other embodiments of the invention, the GLP-1 agonist is Arg34, Lys26(N^{ε}-(γ-GLu(N^{α}-hexadecanoyl)))-GLP-1(7-37).

Since the present invention in part relates to a method of treatment comprising a combination of active agents which may be administered separately or as conjugates, the invention also provides a kit comprising a CD3 agonist and a gastrin compound, a pharmaceutical composition, or conjugate of the invention in kit form. In an aspect, the invention provides a kit for preventing and/or treating diabetes, in particular Type 1 diabetes and LADA, containing a composition comprising a CD3 agonist, and a gastrin compound, a container, and instructions for use. The composition of the kit can further comprise a pharmaceutically acceptable carrier.

These and other aspects, features, and advantages of the present invention should be apparent to those skilled in the art from the following detailed description.

### DETAILED DESCRIPTION OF EMBODIMENTS

In accordance with the present invention there may be employed conventional molecular biology, microbiology, and recombinant DNA techniques within the skill of the art. Such techniques are explained fully in the literature. Sec for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed: 1985); Oligonucleotide Synthesis (M. J. Gaited. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Unless defined otherwise, all technical and scientific terms used herein have the same meaning as commonly understood by one of ordinary skill in the art to which this invention belongs.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 0% or 15%, of the number to which reference is being made. Further, it is to be understood that “a,” “an,” and “the” include plural referents unless the content clearly dictates otherwise. Thus, for example, reference to a composition containing “a compound” includes a mixture of two or more compounds.

Compounds described herein can contain one or more asymmetric centers and may give rise to enantiomers, diasteriomers, and other stereoisomeric forms which may be defined in terms of absolute stereochemistry as (R)- or (S)-. Therefore, the invention includes all such possible diasteriomers and enantiomers as well as their racemic and optically pure forms. Optically active (R)- and (S)-isomers may be prepared using chiral synthons or chiral reagents, or resolved using conventional techniques. When the compounds described herein contain centers of geometric asymmetry, and unless specified otherwise, it is intended that the compounds include both E and A geometric isomers. All tautomeric forms are intended to be included within the scope of the invention.

An “additive effect” of a CD3 agonist and a gastrin compound refers to an effect that is equal to the sum of the effects of the two individual compounds

The term “administering” or “administration” refers to the process by which cell preparations, compositions, CD3 agonists, gastrin compounds, nucleic acids, or chimeric polypeptides are delivered to a subject for treatment or prophylactic purposes. Cell preparations, compositions, CD3 agonists, etc. are administered in accordance with good medical practices taking into account the subject's clinical condition, the site and method of administration, dosage, subject age, sex, body weight, and other factors known to the physician.

An “analog” refers to a polypeptide wherein one or more amino acid residues of a parent or native polypeptide have been substituted by another amino acid residue, one or more amino acid residues of a native or parent polypeptide have been inverted, one or more amino acid residues of the native or parent polypeptide have been deleted, and/or one or more amino acid residues have been added to the parent peptide. Such an addition, substitution, deletion, and/or inversion may be at either of the N-terminal or C-terminal end or within the native or parent polypeptide, or a combination thereof. Typically “an analog” is a peptide wherein 6 or less amino acids have been substituted and/or added and/or deleted from the parent peptide, more preferably a peptide wherein 3 or less amino acids have been substituted and/or added and/or deleted from the parent peptide, and most preferably, a peptide wherein one amino acid has been substituted and/or added and/or deleted from the parent peptide.

Mutations may be introduced into a polypeptide by standard methods, such as site-directed mutagenesis and PCR-mediated mutagenesis. Conservative substitutions can be made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed.

An “antibody” refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, including an antibody fragment. An antibody fragment is a portion of an antibody such as F(ab′)₂, F(ab)₂, Fab′, Fab, Fv, sFv and the like. An antibody fragment binds with the same antigen that is recognized by the intact antibody. An antibody fragment also includes any synthetic or genetically engineered protein that acts like an antibody by binding to a specific antigen to form a complex. For example, an antibody fragment includes an isolated fragment comprising the variable regions of the heavy and light chains (Fv proteins), recombinant single chain polypeptide molecules in which light and heavy variable regions are connected by a peptide linker (“scFv proteins”), and minimal recognition units consisting of the amino acid residues that mimic the hypervariable region. A naked antibody is generally an entire antibody that is not conjugated and includes both polyclonal and monoclonal antibodies, as well as certain recombinant antibodies, such as chimeric, humanized or human antibodies. A chimeric antibody is a recombinant protein that contains the variable domains including the complementarity determining regions (CDRs) of an antibody derived from one species (e.g. rodent antibody) while the constant domains of the antibody molecule is derived from those of a human antibody.

A humanized antibody is a recombinant protein in which the complementarity determining regions (CDRs) from an antibody from one species (e.g., a rodent antibody) is transferred from the heavy and light variable chains of that antibody into human heavy and light variable domains. The constant domains of the antibody are derived from those of a human antibody. Methods for obtaining human antibodies from transgenic mice are described by Green et al., Nature Genet. 7:13 (1994), Lonberg et al., Nature 368:856 (1994), and Taylor et al., int. immun. 6:579 (1994). A fully human antibody also can be constructed by genetic or chromosomal transfection methods, as well as phage display technology, all of which are known in the art. [See McCafferty et al., Nature 348:552-553 (1990) and Johnson and Chiswell, Current Opinion in Structural Biology 3:5564-571 (1993).] Humanized antibodies may also be generated by in vitro activated B cells. [See U.S. Pat. Nos. 5,567,610 and 5,229,275.]

The terms “associated”, “linked”, “interact”, “interaction”, or “interacting” refer to any physical association between molecules. The terms preferably refer to a stable association between two molecules due to, for example, electrostatic, hydrophobic, ionic, hydrogen-bond interactions, or covalent interactions. Certain interacting or associated molecules interact only after one or more of them has been activated.

A “beneficial effect” refers to an effect of a CD3 agonist and preferably a gastrin compound, a composition, conjugate thereof, nucleic acid construct, and/or method of the invention, including favorable pharmacological and/or therapeutic effects, and improved pharmacokinetic properties and biological activity. In aspects of the invention, beneficial effects include but are not limited to the following: reduced or absent islet inflammation, decreased or prevention of disease progression, increased survival, or treatment or reversal of a disease or condition. In other aspects of the invention beneficial effects may be evidenced by reduction in destruction of beta cells, and/or an increase of beta cell function as measured by C-peptide after treatment has been discontinued.

In an embodiment, the beneficial effects can be evidenced in diabetes by one or more of the following: (a) a reduction in fasting blood glucose levels, in particular when blood glucose levels are greater than 7-10 mM; (b) reduction in glycosylated haemoglobin; (c) increase in serum insulin concentration; (d) an increase in beta cell function; (e) an increase in pancreatic insulin production or content; and/or (f) prevention of disease progression. In a particular embodiment, the beneficial effects comprise (a), (b) and (c); (a) (c) and (d); or (a), (c), and (e).

In a preferred embodiment, the beneficial effect is a “sustained beneficial effect” where the beneficial effect is sustained for a prolonged period of time after termination of treatment. A treatment can be sustained over several years thereby having a major beneficial impact on the severity of the disease and its complications

In aspects of the invention, a beneficial effect may be sustained for a prolonged period of at least about 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months, or several years following treatment. The period of time a beneficial effect is sustained may correlate with the duration and timing of the treatment. A subject may be treated continuously for about or at least about 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, or several years, periodically or continuously. In an ex vivo islet neogenesis treatment a subject may be treated with a CD3 agonist, optionally a gastrin compound, and a composition, nucleic acid construct, or conjugate of the invention prior to during or after the transplant of the cells. A sustained beneficial effect may manifest as one or more of increased C-peptide production, increased pancreatic insulin production or concentration, increased beta cell function, and about normal or low blood glucose levels for a prolonged period following treatment. Administration after transplantation can also prolong the function of regenerated cells.

The beneficial effect may be a statistically significant effect in terms of statistical analysis of an effect of a CD3 agonist and gastrin compound and optionally a GLP-1 agonist versus the effects without the compounds or with the individual compounds. “Statistically significant” or “significantly different” effects or levels may represent levels that are higher or lower than a standard. In embodiments of the invention, the difference may be 1.5, 2, 3, 4, 5, or 6 times higher or lower compared with the effect obtained without a CD3 agonist.

A “CD3 agonist” refers to a substance capable of modulating CD3 molecules, and in particular inducing a reaction by acting on CD3 molecules expressed on the surfaces of immunocytes resulting in intracellular signal transduction through CD3, and thereby promoting differentiation of the cells. A CD3 agonist may interact with CD3 and modulate the effects of CD3 or it may be a T cell energizing CD3 agonist. Suitable CD3 agonists include but are not limited to agonistic anti-CD3 antibodies. An anti-CD3 antibody agonist can be selected that reduces or reverses release of cytokines including IL-1, IL-6, IL-2, IL-4 and TNF-α. Such selected anti-CD-3 antibodies include antibodies that have been modified by changing the Fc portion of the immunoglobulin molecule by preparing F(ab′)₂ fragments or modifying the Fc portion of the immunoglobulin to render it non-FcR binding. Examples of anti-CD3 antibodies are OKT3 [ATCC CRL-8001; Orthoclone OKT3™ (Muromonab-CD3), Janssen-Ortho Inc.], UCHT1 (B. D. PharMingen), HIT3a (B. D. PharMingen), hOKT3γl(Ala-Ala), CD3 mAb 145 2Cl 1, YTH 12.5.14.2, YTH 12.5, CAMPATH-3 or F(ab′)₂ fragments thereof. (See for example, see Published PCT WO04106380; Published US Application No. US20040202657; U.S. Pat. No. 6,750,325; U.S. Pat. No. 6,706,265; GB No. 2249310A; and Clark et al., European J. Immunol. 1989, 19:381-388; U.S. Pat. No. 5,968,509.) In an embodiment, a CD3 agonist is a T cell energizing CD3 agonist.

Agonists for various T cell antigen receptors, in particular antibodies having agonistic action, can also be used as a CD3 agonist in the present invention, since these antibodies can cause the formation of complexes of T cell antigen receptors and CD3, resulting in intracellular signal transduction mediated by CD3. A specific example of a CD3 agonist is OT145 which is an antibody for a human T cell antigen receptor Vbeta6.7 (Posnett et al., 1986, Proc. Natl. Acad. Sci. USA., 83(20):7888-92). CD3 agonists may be prepared using conventional processes or they may be obtained from commercial sources. In particular, anti-CD3 mAb may be produced by mammalian cell culture known in the art of mAb production.

A “chimeric polypeptide” comprises all or part (preferably biologically active) of a selected polypeptide (e.g. CD3 agonist or gastrin compound) operably linked to an exogenous polypeptide (i.e. a polypeptide other than the selected polypeptide such as a gastrin compound). Within the fusion protein, the term “operably linked” is intended to indicate that a selected polypeptide and the exogenous polypeptide are fused in-frame to each other. The exogenous polypeptide can be fused to the N-terminus or C-terminus of a selected polypeptide. Chimeric and fusion proteins can be produced by standard recombinant DNA techniques.

A “combination treatment” and “administering in combination” mean that the active ingredients are administered concurrently to a patient being treated. When administered in combination each component may be administered at the same time, or sequentially in any order at different points in time. Therefore, each component may be administered separately, but sufficiently close in time to provide the desired effect, in particular a beneficial, additive, or synergistic effect. The first compound may be administered in a regimen that additionally comprises treatment with the second compound. In aspects the terms refer to the administration of a CD3 agonist, gastrin compound and optionally a GLP-1 agonist within one year, including separate administration of medicaments each containing one of the compounds as well as simultaneous administration whether or not the compounds are combined in one formulation or whether they are in separate formulations.

“Condition(s)” and/or “disease(s)” are used interchangeably or together herein and refer to one or more pathological symptoms or syndromes for which either or both a CD3 agonist and a gastrin compound provide a therapeutic effect. The condition or disease may require reduction of blood glucose levels, inhibition of gastric acid secretion, inhibition of apoptosis of β-cells, stimulation of proliferation or differentiation of β-cells, and reduction of body weight. Examples of conditions and diseases include but are not limited to dyslipidemia, hyperglycemia, severe hypoglycemic episodes, stroke, left ventricular hypertrophy, arrhythmia, bacteremia, septicemia, irritable bowel syndrome, functional dyspepsia, diabetes, catabolic changes after surgery, stress induced hyperglycemia, gastric ulcers, myocardial infarction, impaired glucose tolerance, hypertension, chronic heart failure, fluid retentive states, metabolic syndrome and related diseases and disorders, obesity, diabetic complications as well as symptoms of other diseases in which tissue is damaged due to elevated glucose levels, including Alzheimer's Disease, Parkinson's Disease, and other age-related, tissue-degenerative diseases, as well as the atherogenic effects of elevated leptin, for example in patients with impaired glucose tolerance and obese non-diabetic patients.

A “derivative” refers to a polypeptide in which one or more of the amino acid residues of a native or parent polypeptide or analog thereof have been chemically modified. A chemical modification includes adding chemical moieties, creating new bonds, and removing chemical moieties. A polypeptide may be chemically modified, for example, by alkylation, acylation, glycosylation, pegylation, ester formation, deamidation, amide formation or introducing lipophilic functionalities.

The term “diabetes” as used herein means any manifested symptoms of diabetes in any mammal including experimental animal models, and including human forms such as Type 1 and Type 2 diabetes, LADA, early stage diabetes, and a pre-diabetic condition characterized by mildly decreased insulin or mildly elevated blood glucose levels. A “pre-diabetic condition” describes a subject demonstrating a symptom in terms of insulin or glucose level, and/or demonstrating a susceptibility to diabetes or a related condition due to family history, genetic predisposition, or obesity in the case of Type 2 diabetes, and includes a subject who has previously had diabetes or a related condition and is subject to risk of recurrence.

In aspects of the invention, the condition and/or disease are Type 1 diabetes and LADA. In these aspects, prevention is defined as the management and care of a Type 1 or LADA diabetes patient at diagnosis or later.

“Exogenous gene” refers to a gene expressing a gene product (“exogenous polypeptide”) including but not limited to proteins, peptides, glycoproteins, lipoproteins, and products produced by way of gene replacement to defective organs such as insulin, amylase, protease, lipase, phospholipase, and elastase, gene products produced by the liver including blood clotting factors, UDP glucuronyl transferase, ornithine transcarbanoylase, cytochrome p450 enzymes, adenosine deaminase, gene products produced by the thymus such as serum thymic factor, thymic humoral factor, thymoprotein, and thymosin, and gene products produced by the digestive tract including secretin, cholecystokinin, somatostatin, serotonin, and substance P. In an aspect of the invention the exogenous gene encodes a therapeutic.

A “gastrin compound” is understood to refer to any compound, including peptides and non-peptide compounds, which fully or partially, directly or indirectly, potentiate, induce, mimic, or otherwise enhance the activity of gastrin or a gastrin/CCK receptor in particular a gastrin/CCK_{B} receptor. In particular, a gastrin compound can be used which fully or partially associates and/or activates a gastrin/CCK receptor. A gastrin/CCK receptor includes receptors that associate with gastrin.

In some applications of the invention, a gastrin compound may be a ligand that associates, binds to, interacts with or stimulates a gastrin/CCK receptor, (“gastrin/CCK receptor ligand”). A gastrin compound may be selected that is a peptide or non-peptide small molecule that has a suitable IC₅₀, for example an IC₅₀ of about −0.7 nM at a gastrin/CCK_{B} receptor, as measured by methods known in the art (see Singh et al (1995) J. Biol. Chem. 270: 8429-8438, and Kopin et al (1995) J. Biol. Chem. 270: 5019-5023 describing in vitro cell growth assays, and receptor binding assays as described in Singh et al (1995) J. Biol. Chem. 270: 8429-8438, and Kopin et al (1995) J. Biol. Chem. 270: 5019-5023). A gastrin compound may also be selected based on other criteria such as activity (e.g. synergistic activity with a CD3 agonist), half-life, etc. as discussed herein.

A “gastrin compound” includes native-sequence or synthetic gastrin polypeptides, fragments, analogs (e.g. muteins), derivatives, isoforms, chimeric polypeptides, polypeptides with sequence identity, peptidomimetics, and pharmaceutically acceptable salts thereof, and active metabolites and prodrugs.

A gastrin compound includes, without limitation, the various forms of gastrin, such as gastrin 71, gastrin 52, gastrin 34 (big gastrin), gastrin 17 (little gastrin), gastrin 14, and gastrin 8 (mini gastrin), pentagastrin, tetragastrin and fragments, analogs, and derivatives thereof. Sequences for gastrins including big gastrin-34 (Bonato et al, 1986, Life Science 39:959) and small gastrin-17 (Bentley et al (1966) Nature 209:583) are known in the art, and some are shown in SEQ ID NOs. 11 to 19. In particular, sequences for gastrins include gastrin 71 of SEQ ID NO. 15, gastrin 52 of SEQ ID NO. 16, gastrin 34 (big gastrin) of SEQ ID NO. 11 or 12, gastrin 17 (little gastrin) of SEQ ID NO. 13 or 14, gastrin 14 of SEQ ID NO. 17, and gastrin 6 of SEQ ID NO.18 or 19. Gastrin-34 (big gastrin) is essentially an extension of an amino acid sequence at the N-terminal end of gastrin-17. Big gastrin is cleaved in vivo to release gastrin-17. Glp at the N-terminal end of a gastrin is pyroglutamate, which is a naturally cyclized form of glutamate. In various embodiments, where cysteine or lysine is added to a terminus of gastrin having a pyroglutamate, the pyroglutamate is replaced with a glutamate, or the pyroglutamate is deleted. A gastrin 34 or gastrin-17 may be used in the invention where there is a methionine or a leucine at position 15, as shown in SEQ ID NOs: 11-14 herein.

Examples of gastrin compounds that may be used in the present invention include the compounds disclosed in U.S. Pat. No. 6,288,301. In some applications of the invention, a gastrin compound may be selected that is a peptide or non-peptide agonist or partial agonist of a gastrin/CCK receptor such as A71378(Lin et al., Am. J. Physiol. 258 (4 Pt 1): G648, 1990). In other applications of the invention, a gastrin compound may be a gastrin/CCK receptor ligand including but not limited to gastrin compounds described herein, cholecystokinin (CCK) such as CCK 58, CCK 33, CCK 22, CCK 12 and CCK 8; and the like.

In certain aspects, a gastrin compound may be an active analog, fragment or other modification which, for example, share amino acid sequence identity with an endogenous mammalian gastrin or native-sequence gastrin, for example, share 60%, 70%, 80%, 90%, 95%, 98%, or 99% sequence identity.

Gastrin compounds also include substances that increase the secretion of endogenous gastrins, cholecystokinins or similarly active peptides from sites of tissue storage. Examples of these are the gastric releasing peptide, omeprazole which inhibits gastric acid secretion and increases plasma gastrin levels, soya bean trypsin inhibitor which increases CCK stimulation, and gastrin releasing peptide, which stimulates gastrin secretion without binding to gastrin receptors.

A “gastrin compound” includes a modified form of a gastrin, including but not limited to a modified form of gastrin 71 [SEQ ID NO. 15], gastrin 52 [SEQ ID NO. 16], gastrin 34 (big gastrin) [SEQ ID NO. 11 or 12], gastrin 17 (little gastrin) [SEQ ID NO. 13 or 14], gastrin 14 [SEQ ID NO. 17], gastrin 8, gastrin 6 [SEQ ID NO. 18 and 19], pentagastrin, and tetragastrin. A modified gastrin preferably comprises TrpMetAspPhe-NH₂ [SEQ ID NO. 26] or TrpLcuAspPhe-NH₂ [SEQ ID NO.27].

In aspects of the invention a modified gastrin comprises at least amino acids 1-34, 18-34 or 29-34 of SEQ ID NO. 11 or 12, or amino acids 1-17, 2-17, 12-17, or 14-17 of SEQ ID NO. 13 or 14.

Modified gastrin compounds for use in the present invention comprise the modified gastrin compounds described in PCT/CA03/01778, U.S. Ser. No. 10/719,450 and U.S. Application Ser. No. 60/519,933 incorporated in their entirety by reference. A modified gastrin compound can have an extended activity upon administration to a subject in comparison to a native-sequence gastrin, and they may have a longer half-life in the circulation of a subject.

In particular, a modified gastrin can be a gastrin derivative or analogue comprising a minimal sequence of 6 amino acids (from the C-terminal end) of a gastrin, in particular amino acid residues 1 to 34, 18 to 34 or 29-34 of SEQ ID NO: 11 or 12, or amino acid residues 1-17, 2-17, 12-17, or 14-17 of SEQ ID NO. 13 or 14, and comprising a reactive group capable of undergoing an addition reaction. Examples of reactive groups include without limitation thiols, alpha amino groups, epsilon amino groups, carboxyl groups or aromatic rings. A reactive group is generally capable of linking a gastrin sequence, directly or indirectly via a crosslinking agent and/or spacer region, to a carrier.

A reactive group may be introduced by adding or substituting an amino acid comprising a reactive group, for example by adding a cysteine or lysine. Therefore, a modified gastrin may comprise a gastrin sequence (e.g. gastrin-34 or gastrin 17) wherein at least one reactive amino acid (e.g. cysteine or lysine) is added or substituted. The addition of a reactive amino acid can be at a terminal region, in particular an N-terminal region.

A modified gastrin may also optionally comprise a spacer. A spacer can interact with a reactive group, for example, an amino acid comprising a reactive group. A spacer can be one or more amino acids, peptides, peptidomimetics, or small organic molecules. A spacer can comprise at least one amino acid, preferably at least two, three, four or five amino acids and in certain embodiments it is a sequence of several amino acids, including without limitation alanine or glycine. A spacer can comprise alternating amino acids (e.g. glycine and/or alanine), non-alternating amino acids, a random sequence or a particular sequence. By way of example, a spacer can be synthesized as part of, or may be chemically attached to an amino acid of a gastrin sequence.

A modified gastrin may optionally comprise a cross-linking agent. A cross-linking agent may comprise a homobifunctional or heterobifunctional portion for interaction directly or indirectly with a gastrin, spacer and/or a reactive group. A cross-linking agent may interact with a gastrin sequence or a spacer, or it may be added to a reactive group at the end (in particular N-terminus) of a modified gastrin.

A cross-linking agent can be any agent that can link a gastrin sequence and a carrier directly or via a spacer. Examples of homobifunctional crosslinking agents include without limitation amino group directed homobifunctional cross-linking reagents such as bisamidates (e.g. methyl acetamidate-HCl), bifunctional aryl halides (e.g. 1,5-dichloro-2,4-dinitrobenzene), bifunctional acylating agents (e.g. diisocyanates), bifunctional sulfonyl halides (e.g. phenol-2,4-disulfonyl-chloride), bifunctional acylazides (e.g. tartryl diazide), dialdehydes (e.g. glutaraldehyde), and diketones (e.g. 2,5-hexanedione). Examples of heterobifunctional crosslinkers include amino and sulfhydryl group directed bifunctional reagents (e.g. N-succinimidyl-3-(2-pyridyldithio propionate, carboxyl and either sulfhydryl or amino group directed bifunctional reagents (e.g. p-nitrophenyl diazoacetate), and carbonyl and sulfhydryl group directed bifunctional reagents (e.g. 1-(aminooxy)-4-[3-nitro-2-pyridyl)dithiol)]butane).

A modified gastrin can optionally comprise a carrier which may be a polymer. A carrier may be a polymer of amino acids (proteins), sugars (polysaccharides), nucleosides, synthetic polymers and mixtures thereof. A protein carrier may be a protein found in the circulatory system. Examples of protein carriers found in the circulatory system, in particular the human circulatory system, include without limitation plasma components such as serum, purified serum proteins such as albumin (in particular human serum albumin), transferrin, or an immunoglobulin, red blood cell proteins such as glycophorin A and AE-1, sugar binding proteins such as a lectin, inactivated enzymes, phosphate and sulphate binding proteins, and lipid binding proteins. Examples of other suitable polymeric carriers include without limitation cellulose and derivatives thereof, starch and derivatives thereof, heparin and derivatives thereof, and synthetic polymers such as polyethylene glycol (PEG) and dextran, and derivatives thereof. Carriers may be attached to a gastrin or spacer by way of reactive groups on, or introduced to, the carrier, gastrin, and/or spacer. For example, carriers can be covalently attached to reactive groups (such as thiol groups, alpha and epsilon amino groups, carboxyl groups or aromatic groups) on a gastrin or spacer which may be present or added by chemical modification of the gastrin or spacer.

In certain aspects of the invention, a modified gastrin can comprise a gastrin of SEQ ID NOS 11, 12, 13, 14, 17, or 18 and a carrier.

A group of modified gastrin compounds include compounds having an amino acid sequence comprising from the amino terminus Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr and wherein AA₆ is optionally amidated; Z is a carrier, in particular a polymer and when the polymer is a protein Z is an amino acid sequence; Yₘ is an optional spacer region comprising m amino acid residues of a small neutral amino acid including but not limited to serine and alanine, and X is any consecutive portion of residues 1-28 (=n) of SEQ ID NO: 11 or 12 or 1-11 of SEQ ID. NO. 13 or 14, providing that the gastrin compound binds a gastrin/CCKₐ receptor. Generally, m is 0 to about 20 residues. In an aspect Z is a protein, in particular a protein of the circulatory system, more particularly a serum protein, still more particularly albumin, most particularly human serum albumin.

In embodiments, X is one or more amino acid residues from position 18 to position 28 of SEQ ID NO: 11. Therefore, the gastrin compounds by virtue of the presence of X, can have any of gastrin sequences from positions 18-28, 19-28, 20-28, 21-28, etc. The gastrin compound optionally contains an amino acid spacer (Y) of length m, and m is 0 to about 20 residues.

In embodiments, X is one or more amino acid residues from position 1 to 11 or 2 to 11 of SEQ ID NO: 13 or 14. Therefore, the gastrin compounds by virtue of the presence of X, can have any of gastrin sequences from positions 2 to 11, 3 to 11, 4 to 11, 5 to 11, etc. The gastrin compound optionally contains an amino acid spacer (Y) of length m, and m is 0 to about 20 residues.

A gastrin compound includes a modified gastrin compound of the formula Xₙ-AA₁-AA₂-AA₃-AA₄-AA₅-AA₆, where there is no spacer (Y) and m is 0, which may further comprise a bifunctional cross-linking agent for interaction or linkage to a carrier Z, where Z further comprises a non-proteinaceous polymer such as dextran or PEG.

A modified gastrin compound particularly described herein may further comprise an amino terminal cysteine or lysine residue.

In some embodiments of modified gastrin compounds described herein, the gastrin component contains at least amino acid residues 29-34 of SEQ ID NO: 11 or 12, and it is associated with a polymer, a lipid or a carbohydrate. The polymer may be a synthetic or naturally occurring polymer. The term polymer includes a protein polymer of amino acids, and is not limited to a synthetic polymer. The polymer may be a polyethylene glycol (PEG) or a dextran. A modified gastrin compound can be based on SEQ ID NO: 11 or 12 or “big” gastrin-34 and have a residue at position 32 which is a methionine or a leucine, respectively.

Another preferred modified gastrin compound comprises a structure C-Yₘ-X, wherein C is Cys or Lys, Yₘ is an optional spacer region comprising m amino acid residues of a small neutral amino acid, and X is at least six amino acid residues comprising at least positions 12-17 of gastrin-17 (SEQ ID NO: 13 or 14) or at least positions 29-34 of gastrin-34 (SEQ ID NO: 11 or 12). This modified gastrin compound can further comprise a bifunctional cross-linking agent wherein one reactive portion of the cross-linking agent is covalently linked to C, and the other reactive portion is covalently linked to a polymer or protein.

In a particular aspect of the invention AA₁-AA₂-AA₃-AA₄-AA₅-AA₆ in a modified gastrin compound is Tyr-Gly-Trp-Met-Asp-Phe [SEQ ID NO.23] or Tyr-Gly-Trp-Leu-Asp-Phe [SEQ ID NO.24].

Gastrin compounds may be synthesized using conventional processes. For example, small forms of gastrin such as gastrin 17 are economically prepared by peptide synthesis. In particular, gastrin compounds may be synthesized by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart). The synthesis may be performed using manual procedures or by automation. Automated synthesis may be carried out, for example, using an Applied Biosystems 431A peptide synthesizer (Perkin Elmer). Gastrin compounds can be prepared by recombinant methods well known to those skilled in the art. Thus, the invention contemplates the use of a nucleotide sequence encoding a gastrin compound and optionally a regulatory element, and a host cell comprising the nucleotide sequence for the preparation of a gastrin compound.

Gastrin compounds may also be obtained from commercial sources. For example, synthetic human gastrin 17 with methionine or leucine at position 15 is available from Bachem AG, Bubendorf, (Switzerland), and from Research Plus Inc (New Jersey, USA).

A “gastrin/CCK receptor” refers to a member of the G-protein-coupled receptor family that displays a characteristic binding affinity for a cholecystokinin (CCK) including without limitation CCK-8, desulfated CCK-8, CCK-33, CCK-4, or gastrins including without limitation desulfated or sulfated gastrin-17, or pentagastrin, or other CCK or gastrin analogues or family members. Examples of CCK/gastrin receptor proteins are CCK_{A} and CCK_{B}/gastrin receptors, in particular CCK_{B}/gastrin receptors.

“Gene therapy” refers to the transfer and stable insertion of new genetic information into cells for the therapeutic treatment of a disorder, disease and/or condition described herein. An exogenous gene is transferred into a cell that proliferates to introduce the transferred gene throughout the cell population. In aspects of the invention, an exogenous gene encodes a therapeutic.

Gene therapy can involve one of the following approaches: (i) ex vivo or cellular gene therapy; and (ii) in vivo gene therapy. In ex vivo gene therapy cells are removed from a subject, and while being cultured are treated in vitro. An exogenous gene is introduced into the cells via an appropriate delivery vehicle/method (transfection, transduction, homologous recombination, etc.) and regulatory elements as required, and the modified cells are expanded in culture and returned to the subject. The genetically re-implanted cells express the transfected exogenous gene in situ. In in vivo gene therapy an exogenous gene is introduced into tissues and cells in subjects, for example, by systemic administration or direct injection into sites its situ.

In the present context, “a GLP-1 agonist” is understood to refer to any compound, including peptides and non-peptide compounds, which fully or partially activate the human GLP-1 receptor. In a preferred embodiment, the “GLP-1 agonist” is any peptide or non-peptide small molecule that binds to a GLP-1 receptor, preferably with an affinity constant (K_{D}) or a potency (EC₅₀) of below 1 μM, e.g., below 100 nM, as measured by methods known in the art (see e.g. WO 98/08871) and exhibits insulinotropic activity, where insulinotropic activity may be measured in vivo or in vitro assays known to those of ordinary skill in the art. For example, the GLP-1 agonist may be administered to an animal and the insulin concentration measured over time.

Methods for identifying GLP-1 agonists are described in WO 93/19175 (Novo Nordisk A/S) and examples of suitable GLP-1 analogues and derivatives which can be used according to the present invention includes those referred to in WO 99/43705 (Novo Nordisk A/S), WO 99/43706 (Novo Nordisk A/S), WO 99/43707 (Novo Nordisk A/S), WO 98/08871 (Novo Nordisk A/S), WO 99/43708 (Novo Nordisk A/S), WO 99/43341 (Novo Nordisk A/S), WO 87/06941 (The General Hospital Corporation), WO 90/11296 (The General Hospital Corporation), WO 91/11457 (Buckley et al.), WO 98/43658 (Eli Lilly & Co.), EP 0708179-A2 (Eli Lilly & Co.), EP 0699686-A2 (Eli Lilly & Co.), WO01/98331 (Eli Lilly & Co). Examples of GLP-1 agonists are also listed in Table 1.

In aspects of the invention the GLP-1 agonist is a naturally truncated GLP-1 polypeptide (GLP-1(7-36) or ((GLP-1(7-37)), or an analogue or derivative thereof. The sequences of these naturally occurring truncated GLP-1 agonists are represented in SEQ ID NOs. 4, 5, and 6.

In certain aspects of the invention, a GLP-1 agonist may have the amino acid sequence of SEQ ID NOs. 1, 2, or 3 modified so that amino acid residues at positions 1-20, preferably 1-15, more preferably 1-10, most preferably 1-5 differ from the sequences of SEQ ID NOs. 1, 2 or 3.

In an embodiment of the invention, the GLP-1 agonist is an analogue of GLP-1(7-37) or GLP-1(7-36) which has less than 10 amino acid residues that are different from those in GLP-1(7-37) or GLP-1(7-36), less than 5 amino acid residues that are different from those in GLP-1(7-37) or GLP-1(7-36), less than 3 amino acid residues that are different from those in GLP-1 (7-37) or GLP-1(7-36), preferably only one amino acid residue that is different from sequence of GLP-1(7-37) or GLP-1(7-36).

In one embodiment, the GLP-1 agonist is selected from the group consisting of GLP-1(7-36)-amide, GLP-1(7-37), a GLP-1(7-36)-amide analogue, a GLP-1(7-37) analogue, or a derivative of any of these. GLP-1 agonists that may have specific utility in the present invention include polypeptides where one or more amino acids have been added to the N-terminus and/or C-terminus of GLP-1(7-37) or GLP-1(7-36). Preferably, about one to six amino acids may be added to the N-terminus and/or from about one to eight amino acids may be added to the C-terminus. In certain applications GLP-1 agonists are selected that have up to 39 amino acids. Amino acids at positions 1-6 of an extended GLP-1 agonist may be selected so that they are the same or are conservative substitutions of the amino acid at the corresponding positions of the parent GLP-1 (7-37) or GLP-1(7-36). Amino acids at positions 38-45 of an extended GLP-1 agonist may be selected so that they are the same or conservative substitutions of the amino acids at the corresponding positions of exendin-3 or exendin-4 (SEQ ID NO. 7 and 8 respectively).

In aspects of the invention a GLP-1 agonist is utilized comprising a position 8 analogue wherein the backbone for such analogs or fragments thereof contain an amino acid other than alanine. The amino acid at position 8 may be selected from glycine, valine, leucine, isoleucine, serine, threonine, or methionine.

In an embodiment a GLP-1 agonist is an insulinotropic analogue of GLP-1(1-37), for example, Met⁸-GLP-1(7-37), wherein the alanine in position 8 has been replaced by methionine and the amino acid residues in position 1 to 6 have been deleted, and Arg³⁴-GLP-1(7-37) wherein the valine in position 34 has been replaced with arginine and the amino acid residues in position 1 to 6 have been deleted.

In another embodiment, GLP-1 agonists are selected that have the sequence GLP-1(7-37)OH and GLP-1(7-36) amide, and the corresponding position 8 analogs wherein the backbone for such analogs contains an amino acid other than alanine. The amino acid at position 8 may be selected from glycine, valine, leucine, isoleucine, serine, threonine, or methionine, preferably valine or glycine. The analogs may additionally contain (a) an amino acid at position 22 selected from glutamic acid, lysine, aspartic acid, arginine, and preferably glutamic acid or lysine; (b) an amino acid at position 30 selected from glutamic acid, aspartic acid, serine, or histidine; (c) an amino acid at position 37 selected from lysine, arginine, threonine, glutamic acid, aspartic acid, serine, tryptophan, tyrosine, phenylalanine, or histidine; and/or (d) amino acid at position 27 selected from alanine, lysine, arginine, tryptophan, tyrosine, phenylalanine, or histidine.

A group of GLP-1 analogs and derivatives for use in the present invention comprises the GLP-1 agonists described in U.S. Pat. No. 5,545,618 and US Patent Application Serial No. 20040018975. The analogs include active GLP-1 peptides, 7-34, 7-35, 7-36 and 7-37 having amino acid substitutions at positions 7-10 and/or are truncations at the C-terminus and/or contain various other amino acid substitutions in the basic peptide. Preferred analogs include those with D-amino acid substitutions in the 7 and 8 positions and/or N-alkylated or N-acylated amino acids in the 7 position since they are particularly resistant to degradation in vivo.

In aspects of the invention, a GLP-1 agonist comprises a peptide comprising or selected from the group consisting of GLP-1 (1-38); GLP-1 (1-39), GLP-1 (1-40), GLP-1 (1-41), GLP-1 (7-38), GLP-1 (7-39), GLP-1 (7-40), and GLP-1 (7-41).

In another aspect of the invention at least one amino acid of a GLP-1 agonist has at least one substituent attached directly or indirectly (e.g. via a spacer such as γ-Glu or β-Ala). A substituent is generally selected to make the profile of action of the parent GLP-1 agonist more protracted, make the GLP-1 agonists more metabolically and physically stable, and/or increase solubility of the GLP-1 agonist. An example of a particular substituent is an amide, a carbohydrate, and a lipophilic substituent.

In one embodiment, the GLP-1 agonist is a derivative of GLP-1(7-36)-amide, GLP-1(7-37), a GLP-1(7-36)-amide analogue or a GLP-1(7-37) analogue, which comprises a lipophilic substituent.

In a particular embodiment of the invention, the GLP-1 derivative preferably has three lipophilic substituents, more preferably two lipophilic substituents, and most preferably one lipophilic substituent attached to the parent peptide (i.e., GLP-1(7-36)-amide, GLP-1(7-37), a GLP-1(7-36)-amide analogue or a GLP-1(7-37) analogue), where each lipophilic substituent(s) preferably has 4-40 carbon atoms, more preferably 8-30 carbon atoms, even more preferably 8-25 carbon atoms, even more preferably 12-25 carbon atoms, and most preferably 14-18 carbon atoms.

In one embodiment, the lipophilic substituent comprises a partially or completely hydrogenated cyclopentanophenathrene skeleton.

In another embodiment, the lipophilic substituent is a straight-chain or branched alkyl group.

In yet another embodiment, the lipophilic substituent is an acyl group of a straight-chain or branched fatty acid. Preferably, the lipophilic substituent is an acyl group having the formula CH₃(CH₂)ₙCO—, wherein n is an integer from 4 to 38, preferably an integer from 12 to 38, and most preferably is CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—. In a more preferred embodiment, the lipophilic substituent is tetradecanoyl. In a most preferred embodiment, the lipophilic substituent is hexadecanoyl.

In a further embodiment of the present invention, the lipophilic substituent has a group which is negatively charged such as a carboxylic acid group. For example, the lipophilic substituent may be an acyl group of a straight-chain or branched alkane a,w-dicarboxylic acid of the formula HOOC(CH₂)ₘCO—, wherein m is an integer from 4 to 38, preferably an integer from 12 to 38, and most preferably is HOOC(CH₂)₁₄CO—, HOOC(CH₂)₁₆CO—, HOOC(CH₂)₁₈CO—, HOOC(CH₂)₂₀CO— or HOOC(CH₂)₂₂CO—.

In the GLP-1 derivatives of the invention, the lipophilic substituent(s) contain a functional group which can be attached to one of the following functional groups of an amino acid of the parent GLP-1 peptide:

(a) the amino group attached to the alpha-carbon of the N-terminal amino acid,

(b) the carboxy group attached to the alpha-carbon of the C-terminal amino acid,

(c) the epsilon-amino group of any Lys residue,

(d) the carboxy group of the R group of any Asp and Glu residue,

(e) the hydroxy group of the R group of any Tyr, Ser and Thr residue,

(f) the amino group of the R group of any Trp, Asn, Gln, Arg, and His residue, or

(g) the thiol group of the R group of any Cys residue.

In one embodiment, a lipophilic substituent is attached to the carboxy group of the R group of any Asp and Glu residue.

In another embodiment, a lipophilic substituent is attached to the carboxy group attached to the alpha-carbon of the C-terminal amino acid.

In a most preferred embodiment, a lipophilic substituent is attached to the epsilon-amino group of any Lys residue.

In a preferred embodiment of the invention, the lipophilic substituent is attached to the parent GLP-1 peptide by means of a spacer. A spacer must contain at least two functional groups, one to attach to a functional group of the lipophilic substituent and the other to a functional group of the parent GLP-1 peptide.

In one embodiment, the spacer is an amino acid residue except Cys or Met, or a dipeptide such as Gly-Lys. For purposes of the present invention, the phrase “a dipeptide such as Gly-Lys” means any combination of two amino acids except Cys or Met, preferably a dipeptide wherein the C-terminal amino acid residue is Lys, His or Trp, preferably Lys, and the N-terminal amino acid residue is Ala, Arg, Asp, Asn, Gly, Glu, Gln, Ile, Leu, Val, Phe, Pro, Ser, Tyr, Thr, Lys, His and Trp. Preferably, an amino group of the parent peptide forms an amide bond with a carboxylic group of the amino acid residue or dipeptide spacer, and an amino group of the amino acid residue or dipeptide spacer forms an amide bond with a carboxyl group of the lipophilic substituent.

Preferred spacers are lysyl, glutamyl, asparagyl, glycyl, beta-alanyl and gamma-aminobutanoyl, each of which constitutes an individual embodiment. Most preferred spacers are glutamyl and beta-alanyl. When the spacer is Lys, Glu or Asp, the carboxyl group thereof may form an amide bond with an amino group of the amino acid residue, and the amino group thereof may form an amide bond with a carboxyl group of the lipophilic substituent. When Lys is used as the spacer, a further spacer may in some instances be inserted between the e-amino group of Lys and the lipophilic substituent. In one embodiment, such a further spacer is succinic acid which forms an amide bond with the e-amino group of Lys and with an amino group present in the lipophilic substituent. In another embodiment such a further spacer is Glu or Asp which forms an amide bond with the e-amino group of Lys and another amide bond with a carboxyl group present in the lipophilic substituent, that is, the lipophilic substituent is a N^{ε}-acylated lysine residue.

In another embodiment, the spacer is an unbranched alkane a,w-dicarboxylic acid group having from 1 to 7 methylene groups, which spacer forms a bridge between an amino group of the parent peptide and an amino group of the lipophilic substituent. Preferably, the spacer is succinic acid.

In a further embodiment, the lipophilic substituent with the attached spacer is a group of the formula CH₃(CH₂)ₚNH—CO(CH₂)_{q}CO—, wherein p is an integer from 8 to 33, preferably from 12 to 28 and q is an integer from 1 to 6, preferably 2.

In a further embodiment, the lipophilic substituent with the attached spacer is a group of the formula CH₃(CH₂)ᵣCO—NHCH(COOH)(CH₂)ₙCO—, wherein r is an integer from 4 to 24, preferably from 10 to 24.

In a further embodiment, the lipophilic substituent with the attached spacer is a group of the formula CH₃(CH₂)ₛCO—NHCH((CH₂)₂COOH)CO—, wherein s is an integer from 4 to 24, preferably from 10 to 24.

In a further embodiment, the lipophilic substituent is a group of the formula COOH(CH₂)ₙCO— wherein t is an integer from 6 to 24.

In a further embodiment, the lipophilic substituent with the attached spacer is a group of the formula —NHCH(COOH)(CH₂)₄NH—CO(CH₂)_{w}CH₃, wherein u is an integer from 8 to 18.

In a further embodiment, the lipophilic substituent with the attached spacer is a group of the formula CH₃(CH₁)ᵥCO—NH—(CH₂)_{z}—CO, wherein v is an integer from 4 to 24 and z is an integer from 1 to 6.

In a further embodiment, the lipophilic substituent with the attached spacer is a group of the formula —NHCH(COOH)(CH₂)₄NH—COCH((CH₂)₂COOH)NH—CO(CH₂)ₙCH₃, wherein w is an integer from 10 to 16.

In a further embodiment, the lipophilic substituent with the attached spacer is a group of the formula —NHCH(COOH)(CH₁)₄NH—CO(CH₂)ₙCH(COOH)NHCO(CH₁)ₓCH₃, wherein x is zero or an integer from 1 to 22, preferably 10 to 16.

In yet another embodiment the GLP-1 agonist is Arg³⁴, Lys²⁶(N^{ε}-(γ-Glu(N^{α}-hexadecanoyl)))-GLP-1(7-37).

In yet another embodiment the GLP-1 agonist is selected from the group consisting of Gly⁸-GLP-1(7-36)-amide, Gly⁸-GLP-1(7-37), Val⁸-GLP-1(7-36)-amide, Val⁸-GLP-1(7-37), Val⁸Asp²²-GLP-1(7-36)-amide, Val⁸Asp²²-GLP-1(7-37), Val⁸Glu²²-GLP-1(7-36)-amide, Val⁸Glu²²-GLP-1(7-37), Val⁸Lys²²-GLP-1(7-36)-amide, Val⁸Lys²²-GLP-1(7-37), Val⁸Arg²²-GLP-1(7-36)-amide, Val⁸Arg²²-GLP-1(7-37), Val⁸His²²-GLP-1(7-36)-amide, Val⁸His²²His²²-GLP-1(7-37), analogues thereof and derivatives of any of these.

In yet another embodiment the GLP-1 agonist is selected from the group consisting of Arg²⁶-GLP-1(7-37); Arg³⁴-GLP-1(7-37); Lys³⁶-GLP-1(7-37); Arg^{26,34}-Lys³⁶-GLP-1(7-37); Arg^{26,34}-GLP-1(7-37); Arg^{26,34}Lys⁴⁰-GLP-1(7-37); Arg²⁶Lys³⁶-GLP-1(7-37); Arg³⁴Lys³⁶-GLP-1(7-37); Val⁸Arg²²-GLP-1(7-37); Met⁸Arg²²-GLP-1(7-37); Gly⁸His²²-GLP-1(7-37); Val⁸His²²-GLP-1(7-37); Met⁸His²²-GLP-1(7-37); His³⁷-GLP-1(7-37); Gly⁸-GLP-1(7-37); Val⁸-GLP-1(7-37); Met⁸-GLP-1(7-37); Gly⁸Asp²²-GLP-1(7-37); Val⁸Asp²²-GLP-1(7-37); Met⁸Asp²²-GLP-1(7-37); Gly⁸Glu²²-GLP-1(7-37); Val⁸Glu²²-GLP-1(7-37); Met⁸Glu²²-GLP-1(7-37); Gly⁸Lys²²-GLP-1(7-37); Val⁸Lys²²-GLP-1(7-37); Met⁸Lys²²-GLP-1(7-37); Gly⁸Arg²²-GLP-1(7-37); Val⁸Lys²²His³⁷-GLP-1(7-37); Gly⁸Glu²²His³⁷-GLP-1(7-37); Val⁸Glu²²His³⁷-GLP-1(7-37); Met⁸Glu²²His³⁷-GLP-1(7-37); Gly⁸Lys²² His³⁷-GLP-1(7-37); Met⁸Lys²²His³⁷-GLP-(7-37); Gly⁸Arg²²His³⁷-GLP-1(7-37); Val⁸Arg²²His³⁷-GLP-1(7-37); Met⁸Arg²²His³⁷-GLP-1(7-37); Gly⁸His²²His³⁷-GLP-1(7-37); Val⁸His²²His³⁷-GLP-1(7-37); Met⁸His²²His³⁷-GLP-1(7-37); Gly⁸His³⁷-GLP-1(7-37); Val⁸His³⁷-GLP-1(7-37); Met⁸His³⁷-GLP-1(7-37); Gly⁸Asp²² His³⁷-GLP-1(7-37); Val⁸Asp²²His³⁷-GLP-1(7-37); Met⁸Asp²²His³″-GLP-1(7-37); Arg²⁶-GLP-1(7-36)-amide; Arg³⁴-GLP-1(7-36)-amide; Lys³⁶-GLP-1(7-36)-amide; Arg^{26,34}Lys³⁶-GLP-1(7-36)-amide; Arg^{26,34}-GLP-1(7-36)-amide; Arg^{26,34} Lys⁴⁰-GLP-1(7-36)-amide; Arg 26Lys¹⁶-GLP-1(7-36)-amide; Arg³⁴Lys³⁶-GLP-1(7-36)-amide; Gly⁸-GLP-1(7-36)-amide; Val⁸-GLP-1 (7-36)-amide; Met⁸-GLP-1 (7-36)-amide; Gly⁸Asp²²-GLP-1(7-36)-amide; Gly⁸Glu²²His³⁷-GLP-1(7-36)-amide; Val⁸Asp²²-GLP-1(7-36)-amide; Met⁸Asp²²-GLP-1(7-36)-amide; Gly⁸Glu²²-GLP-1 (7-36)-amide; Val⁸Glu²²-GLP-1(7-36)-amide; Met⁸Glu²²-GLP-1(7-36)-amide; Gly⁸Lys²²-GLP-1(7-36)-amide; Val⁸Lys²²-GLP-1(7-36)-amide; Met⁸Lys²²-GLP-1(7-36)-amide; Gly⁸His²²His³⁷-GLP-1 (7-36)-amide; Gly⁸Arg²²-GLP-1(7-36)-amide; Val⁸Arg²²-GLP-1(7-36)-amide; Met⁸Arg²²-GLP-1(7-36)-amide; Gly⁸His²²-GLP-1(7-36)-amide; Val⁸His²²-GLP-1(7-36)-amide; Met⁸His²²-GLP-1(7-36)-amide; His³⁷-GLP-1(7-36)-amide; Val⁸Arg²²His³⁷-GLP-1(7-36)-amide; Met⁸Arg²²His³⁷-GLP-1(7-36)-amide; Gly⁸His³⁷-GLP-1(7-36)-amide; Val⁸His³⁷-GLP-1(7-36)-amide; Met⁸His³⁷-GLP-1(7-36)-amide; Gly⁸Asp²² His³⁷-GLP-1(7-36)-amide; Val⁸Asp²²His³⁷-GLP-1(7-36)-amide; Met⁸Asp²²His³⁷-G LP-1 (7-36)-amide; Val⁸Glu²²His³⁷-GLP-1 (7-36)-amide; Met⁸Glu²²His³⁷-GLP-1(7-36)-amide; Gly⁸Lys²² His³⁷-GLP-1(7-36)-amide; Val⁸Lys²²His³⁷-GLP-1(7-36)-amide; Met⁸Lys²² His³⁷-GLP-1(7-36)-amide; Gly⁸Arg²²His³⁷-GLP-1 (7-36)-amide; Val⁸His²²His³⁷-GLP-1(7-36)-amide; Met⁸His²² His³⁷-GLP-1(7-36)-amide; and derivatives thereof.

In yet another embodiment the GLP-1 agonist is selected from the group consisting of Val⁸Trp¹⁹Glu²²-GLP-1(7-37), Val⁸Glu²²Val²⁵-GLP-1(7-37), Val⁸Tyr¹⁶Glu²²-GLP-1(7-37), Val⁸Trp¹⁶Glu²²-GLP-1(7-37), Val⁸Leu¹⁶Glu²²-GLP-1(7-37), Val⁸Tyr¹⁸Glu²²-GLP-1(7-37), Val⁸Glu²²His³⁷-GLP-1(7-37), Val⁸Glu²²Ile³³-GLP-1(7-37), Val⁸Trp 16Glu²²Val²⁵Ile³³-GLP-1(7-37), Val⁸Trp¹⁶Glu²²Ile³³-GLP-1(7-37), Val⁸Glu²²Val²⁵Ile³³-GLP-1(7-37), Val⁸Trp¹⁶Glu²²Val²⁵-GLP-1(7-37), analogues thereof and derivatives of any of these.

In a particular embodiment, the GLP-1 agonist comprises or is selected from the group consisting of Gly⁸-GLP-1(7-37), Val⁸GLP-1(7-37), Val⁸Asp²²GLP-1(7-37), Val⁸Glu²²GLP-1(7-37), Val⁸Lys²²GLP-1(7-37), and Val⁸His²² GLP-1(7-37), and analogs and derivatives thereof.

In a further particular embodiment, the GLP-1 agonist comprises or is selected from the group consisting of Gly⁸-GLP-1(7-36) amide, Val⁸GLP-1(7-36) amide, Val⁸Asp²²GLP-1(7-36)amide, Val⁸Glu²²GLP-1(7-36) amide, Val⁸Lys²²GLP-1(7-36) amide, and Val⁸His²² GLP-1(7-36) amide, and analogs and derivatives thereof.

In yet another embodiment the GLP-1 agonist is a stable GLP-1 analogue/derivative. Throughout this application a “stable GLP-1 analogue/derivative” means a GLP-1 analogue or a derivative of a GLP-1 analogue which exhibits an in vivo plasma elimination half-life of at least 10 hours in man, as determined by the method described below. Examples of stable GLP-1 analogue/derivatives can be found in WO 98/08871 and WO 99/43706. The method for determination of plasma elimination half-life of a compound in man is: The compound is dissolved in an isotonic buffer, pH 7.4, PBS or any other suitable buffer. The dose is injected peripherally, preferably in the abdominal or upper thigh. Blood samples for determination of active compound are taken at frequent intervals, and for a sufficient duration to cover the terminal elimination part (e.g. Pre-dose, 1, 2, 3, 4, 5, 6, 7, 8, 10, 12, 24 (day 2), 36 (day 2), 48 (day 3), 60 (day 3), 72 (day 4) and 84 (day 4) hours post dose). Determination of the concentration of active compound is performed as described in Wilken et al., Diabetologia 43(51):A143, 2000. Derived pharmacokinetic parameters are calculated from the concentration-time data for each individual subject by use of non-compartmental methods, using the commercially available software WinNonlin Version 2.1 (Pharsight, Cary, N.C., USA). The terminal elimination rate constant is estimated by log-linear regression on the terminal log-linear part of the concentration-time curve, and used for calculating the elimination half-life.

Stable GLP-1 analogues and derivatives are disclosed in WO 98/08871 (analogues with lipophilic substituent) and in WO 02/46227 (analogues fused to serum albumin or to Fc portion of an Ig).

In another embodiment, the GLP-1 agonist is formulated so as to have a half-life in man, as discussed above, of at least 10 hours. This may be obtained by sustained release formulations known in the art.

In yet another embodiment the GLP-1 agonist is exendin-4 or exendin-3, an exendin-4 or exendin-3 analogue or a derivative of any of these.

Examples of exendins as well as analogues, derivatives, and fragments thereof to be included within the present invention are those disclosed in WO97/46584, U.S. Pat. No. 5,424,286 and WO01/04156. U.S. Pat. No. 5,424,286 describes a method for stimulating insulin release with an exendin polypeptide. The exendin polypeptides disclosed include HGEGTFTSDLSKQMEEEAVRLFIEWLKNGGX [SEQ ID NO. 20]; wherein X=P or Y, and HX1X2GTFITSDLSKQMEEEAVPLFIEWLKNGGPSSGAPPPS [SEQ ID NO. 21]; wherein X1X2=SD (exendin-3) or GE (exendin-4)). WO 97/46584 describes truncated versions of exendin peptide(s). The disclosed peptides increase secretion and biosynthesis of insulin, but reduce those of glucagon. WO 01/04156 describes exendin-4 analogues and derivatives as well as the preparation of these molecules. Exendin-4 analogues stabilized by fusion to serum albumin or Fe portion of an Ig are disclosed in WO 02/46227.

In one embodiment, the exendin-4 analogue is HGEGTFTSDLSKQMEEEAVRLFIEWLKNGGPSSGAPPSKKKKKK [SEQ ID NO. 22].

In yet another embodiment the GLP-1 agonist is a stable exendin-4 analogue/derivative. The term “stable exendin-4 analogue/derivative”, as used herein refers to an exendin-4(1-39) analogue or a derivative of an exendin-4(1-39) analogue which exhibits an in vivo plasma elimination half-life of at least 10 hours in man, as determined by the method described above for a “stable GLP-1 analogue/derivative”.

In still another embodiment, the GLP-1 agonist is Aib^{8,35} GLP-1(7-36) amide (Aib=a-amino isobutyric acid).

In still another embodiment, the GLP-1 agonist is Ser³⁸,Lys^{39,40,41,42,43,44}-Exendin-4(1-39)amide.

In still another embodiment the GLP-1 agonist is selected from the non-peptide small molecule GLP-1 agonists disclosed in WO 00/42026.

An amino acid portion of a GLP-1 agonist can be prepared by a variety of methods known in the art such as solid-phase synthesis, purification of GLP-1 agonists from natural sources, recombinant technology, or a combination of these methods. See for example, U.S. Pat. Nos. 5,188,666, 5,120,712, 5,523,549, 5,512,549, 5,977,071, 6,191,102, Dugas and Penney 1981, Merrifield, 1962, Stewart and Young 1969, and the references cited herein. GLP-1 agonist derivatives can be produced by appropriate derivatization of an appropriate backbone produced, for example, by recombinant DNA technology or peptide synthesis (e.g. Merrifield-type solid phase synthesis) using methods known in the art of peptide synthesis and peptide chemistry.

“Host cells” comprising a nucleotide sequence of a CD3 agonist and optionally a gastrin compound or a GLP-1 agonist, or a nucleic acid construct include a wide variety of prokaryotic and eukaryotic host cells. For example, the polypeptides may be expressed in bacterial cells such as E. coli, Bacillus, or Streptomyces, insect cells (using baculovirus), yeast cells, or mammalian cells. Other suitable host cells can be found in Goeddel, Gene Expression Technology: Methods in Enzymology 185, Academic Press, San Diego, Calif. (1991). A host cell may also be chosen which modulates the expression of an inserted nucleotide sequence, or modifies (e.g. glycosylation or phosphorylation) and processes (e.g., cleaves) the polypeptide in a desired fashion. Host systems or cell lines may be selected which have specific and characteristic mechanisms for post-translational processing and modification of proteins. For long-term high-yield stable expression of the protein, cell lines and host systems which stably express the gene product may be engineered.

In aspects of the invention, the host cells are mammalian cells. Mammalian host cells may be obtained from pancreatic islets, liver, bone marrow, umbilical cord, embryos, or stem cell lines. In particular aspects, the host cells are stem cells (e.g., from the pancreas or other tissues), islet precursor cells, mature insulin secreting cells or pancreatic insulin secreting β cells.

“Insulinotropic activity” refers to an ability of a substance to stimulate insulin secretion in response to elevated glucose levels, to produce or increase glucose uptake by cells, and decreased serum glucose or blood glucose levels. Methods known in the art can be employed to assay for insulinotropic activity. For example, in vitro and in vivo methods may be used that measure insulin or C-peptide levels. Compounds, compositions, constructs, or conjugates described herein have insulinotropic activity if islet cells secrete insulin in the presence of the compounds, compositions, constructs, or conjugates above background levels or levels in the absence of the CD3 agonists or compositions.

“Islet neogenesis” means formation of new pluripotent pancreatic precursor cells, pancreatic islet precursor cells, or beta cells by proliferation and differentiation, which may or may not have the characteristics of stem cells which have the ability to reproduce in an unlimited manner.

“Modified islet precursor cells”, “modified stem cells”, or “modified cells” refers to a cell into which exogenous genetic material (e.g. a nucleic acid construct or vector of the invention) has been operatively incorporated into its genome, or into which a CD3 agonist and a gastrin compound, conjugate, (e.g., chimeric polypeptide), or compositions have been introduced. In an aspect a modified cell of the invention has a stably incorporated nucleic acid construct, expression of which results in a 2 to 100 fold, 10 to 100 fold, 10 to 50 fold expansion of a pancreatic islet beta cell population.

A “native-sequence polypeptide” or “a native polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc include but are not limited to saline, buffered saline, dextrose, water, glycerol, ethanol, and combinations thereof. The use of such media and agents for an active substance is well known in the art.

“Pharmaceutically acceptable salt(s),” includes salts of acidic or basic groups which may be present in the compounds suitable for use in the present invention. Examples of pharmaceutically acceptable salts include sodium, calcium, ammonium, ferric hydroxides, isopropylamine, triethylamine, 2-ethylamine, 2-ethylamino, ethanol, histidine, procarine, and potassium salts of carboxylic acid groups and hydrochloride salts of amino groups. Other pharmaceutically acceptable salts of amino groups are hydrobromide, sulfate, hydrogen sulfate, phosphate, acetate, oxalic, hydrogen phosphate, dihydrogen phosphate, acetate, succinate, citrate, tartrate, lactate, mandelate, methanesulfonate (mesylate) and p-toluenesulfonate (tosylate) salts.

A “polypeptide variant” as used herein refers to a polypeptide having at least about 60%, 65%, 70%, 75%, 80%, 85%, 90%, 95%, 97%, 98%, or 99% amino acid sequence identity, particularly at least about 70-80%, more particularly at least about 85%, still more particularly at least about 90%, most particularly at least about 95% amino acid sequence identity with a native-sequence polypeptide. Such variants include for instance polypeptides wherein one or more amino acid residues are added to, or deleted from the N- or C-terminus of the full-length or mature sequences of the polypeptide, including variants from other species, but excludes a native-sequence polypeptide. In aspects of the invention variants retain the immunogenic activity of the corresponding native-sequence polypeptide. A naturally occurring allelic variant may contain conservative amino acid substitutions from the native polypeptide sequence or it may contain a substitution of an amino acid from a corresponding position in a polypeptide homolog, for example, a murine polypeptide.

Identity of two amino acid sequences, or of two nucleic acid sequences is generally defined as the percentage of amino acid residues or nucleotides in a candidate sequence that are identical with the amino acid residues in a polypeptide or nucleotides in a nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST programs are publicly available from NCBI and other sources (BLAST Manual, Altschul, S. et al. NCBI NLM NIH Bethesda, Md. 20894; Altschul, S. et al. J. Mol. Biol. 215:403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

The term “treating” refers to reversing, alleviating, or inhibiting the progress of a condition and/or disease, or one or more symptoms of such condition and/or disease, to which such term applies. Depending on the condition of the subject, the term also refers to preventing a condition and/or disease, and includes preventing the onset, or preventing the symptoms associated with a condition and/or disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition of the present invention to a subject that is not at the time of administration afflicted with the disease. “Preventing” also refers to preventing the recurrence of a disease, or of one or more symptoms associated with such disease. The terms “treatment” and “therapeutically,” refer to the act of treating, as “treating” is defined above. The purpose of prevention and intervention is to combat the disease, condition, or disorder and includes the administration of the active compounds to prevent or delay the onset of the symptoms or complications, or alleviating the symptoms or complications, or eliminating the disease, condition, or disorder.

“Regulatory element” refers to a genetic element or elements having a regulatory role in gene expression, for example, promoters or enhancers, Large numbers of suitable vectors and promoters are known to those of skill in the art and are commercially available for generating recombinant constructs encoding a CD3 agonist and optionally a gastrin compound and/or exogenous gene. The following vectors are provided by way of example. Bacterial: pBs, phagescript, PsiX174, pBluescript SK, pBs KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTL pSG (Stratagene) pSVK3, pBPV, pMSG, pSVL (Pharmacia). As defined herein “operably linked” means that an isolated polynucleotide and a regulatory element are situated within a vector or cell in such a way that the polypeptide is expressed by a host cell which has been transformed (transfected) with the ligated polynucleotide/regulatory element sequence. A regulatory element can be a constitutive or induced transcriptional regulatory region, for example, a transcriptional regulatory region from an insulin gene that is induced by increasing intracellular glucose concentrations.

“Inducible regulatory element” refers to a regulatory element that induces expression of a gene to which it is operably linked in response to particular stimuli such as chemicals, chemo-attractants, particular ligands, and the like. An inducible regulatory element (e.g., an inducible promoter) permits modulation of the production of a gene product in a cell. Examples of suitable inducible regulatory systems for use in eukaryotic cells include hormone-regulated elements (e.g., see Mader, S, and White, J. H. (1993) Proc. Natl. Acad. Sci. USA 90: 5603-5607), synthetic ligand-regulated elements (see, e.g., Spencer, D. M. et al 1993) Science 262: 1019-1024) and ionizing radiation-regulated elements (e.g., see Manome, Y. Et al. (1993) Biochemistry 32:10607-10613; Datta, R. et al. (1992) Proc. Natl. Acad. Sci. USA 89: 1014-10153). Additional tissue-specific or inducible regulatory systems, which may be developed, can also be used in accordance with the invention.

The terms “subject”, “individual”, “recipient” or “patient” refer to an animal including a warm-blooded animal such as a mammal, which is afflicted with or suspected of having or being pre-disposed to a a condition and/or disease described herein. Mammal includes without limitation any members of the Mammalia. In general, the terms refer to a human. The terms also include domestic animals bred for food or as pets, including horses, cows, sheep, poultry, fish, pigs, cats, dogs, and zoo animals, goats, apes (e.g. gorilla or chimpanzee), and rodents such as rats and mice. The methods herein for use on subjects/individuals/patients contemplate prophylactic as well as curative use. Typical subjects for treatment include persons susceptible to, suffering from or that have suffered a condition and/or disease described herein. In embodiments of the invention, the subject is suspected of having or has been diagnosed with Type 1 diabetes or LADA.

“Suboptimal dose” or suboptimal dosage” refers to a dose or dosage of an active compound which is less than the optimal dose or dosage for that compound when used in monotherapy.

A “synergistic effect” of a CD3 agonist and a gastrin compound refers to an effect that is greater than the additive effect that results from the sum of the effects of the two individual compounds.

“Test substance” includes but is not limited to proteins, peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], nucleic acids, ribozymes, carbohydrates, and small organic or inorganic molecules. A test substance may be an endogenous physiological compound or it may be a natural or synthetic compound.

A “therapeutic” includes without limitation a peptide, polypeptide, an enzyme, an enzyme inhibitor, an antigen, an antibody, a hormone, a factor involved in cell intrinsic pathways, an interferon, a cytokine, a chemokine, an endocrine hormone (e.g. insulin), a trophic protein, a growth factor, or a tumor toxic protein.

“Therapeutically effective amount” relates to the amount or dose of an active compound (e.g. CD3 agonist or gastrin compound), conjugate, nucleic acid, composition, or cell preparation of the invention that will lead to one or more desired beneficial effects, in particular, one or more sustained beneficial effects. A therapeutically effective amount of a substance can vary according to factors such as the disease state, age, sex, and weight of the individual, and the ability of the substance to elicit a desired response in the individual. Dosage regimens may be adjusted to provide the optimum therapeutic response (e.g. sustained beneficial effects). For example, several divided doses may be administered daily or the dose may be proportionally reduced as indicated by the exigencies of the therapeutic situation.

“Transplanting”, “transplantation”, “grafting” and “graft” are used to describe the process by which cells, preparations, and compositions of the invention are delivered to the site within the patient where the cells are intended to exhibit a beneficial effect, such as treating a condition and/or disease, injury or trauma, or genetic damage. Cells, preparations, and compositions may also be delivered in a remote area of the body by any mode of administration relying on cellular migration to the appropriate area in the body to effect transplantation.

### Nucleic Acid Constructs, Vectors and Host Cells

The invention provides a nucleic acid construct comprising a nucleic acid sequence encoding a mammalian CD3 agonist operably linked to a regulatory element (e.g. a heterologous promoter) and a nucleic acid sequence encoding a mammalian gastrin compound operably linked to a regulatory element (e.g. heterologous promoter). A nucleic acid construct may additionally comprise a sequence encoding an exogenous polypeptide, in particular a sequence encoding a GLP-1 agonist. The invention also contemplates a pharmaceutical composition comprising a nucleic acid construct of the invention.

Nucleic acid constructs of the invention may be chemically synthesized using standard techniques. Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

A nucleic acid construct may be inserted into an appropriate expression vector i.e. a vector that contains the necessary regulatory elements for the transcription and translation of the inserted coding sequences. Accordingly, vectors adapted for transformation of a host cell may be constructed which comprise a nucleic acid construct of the invention and one or more regulatory elements. Vectors that express a nucleic acid construct can be prepared using techniques well known to those skilled in the art (see for example, Sambrook et al.). Possible expression vectors include but are not limited to cosmids, plasmids, or modified viruses (e.g. replication defective retroviruses, adenoviruses and adeno-associated viruses), so long as the vector is compatible with the host cell used. Selection of appropriate regulatory elements is dependent on the host cell chosen as discussed below, and may be readily accomplished by one of ordinary skill in the art. The necessary regulatory elements may be supplied by the native sequence and/or its flanking regions.

A vector can be used to prepare transformed host cells expressing a chimeric polypeptide comprising a CD3 agonist and a gastrin compound and optionally an exogenous polypeptide or a GLP-1 agonist. Therefore, the invention provides host cells comprising or transformed with a construct or vector of the invention. A host cell may be modified by any means known in the art which results in stable integration and expression of a nucleic acid construct or vector in the modified cell and its progeny.

Nucleic acid constructs can be introduced in cells via conventional techniques, such as the methods for transforming and transfecting cells found in Sambrook et al., supra, and other laboratory textbooks. By way of example, a nucleic acid construct may be introduced into host cells using an appropriate expression vector of the invention. Transfection is easily and efficiently obtained using standard methods including culturing the cells on a monolayer of virus-producing cells. Non-viral methods can also be used to cause expression of a CD3 agonist and optionally a gastrin compound or chimeric polypeptide in cells. Most non-viral methods of gene transfer rely on normal mechanisms used by mammalian cells for the uptake and transport of macromolecules. Non-viral methods include but are not limited to calcium phosphate or calcium chloride co-precipitation, DEAE-dextran-mediated transfection, lipofection, electroporation, or microinjection, liposomal derived systems, poly-lysine conjugates, and artificial viral envelopes.

Transduction of cells in vitro may be accomplished by the direct co-culture of cells (e.g., stem cells or islet precursor cells) with producer cells, following methods known in the art. For clinical applications, transduction by culturing the cells with viral supernatant alone or with purified viral preparations is preferred. Polycations, such as protamine sulfate, polybrene and the like, will generally be included to promote binding. Protamine sulfate and polybrene are typically used in the range of 4 μg/ml. Additionally, cytokines may also be added, including, e.g., IL-3, IL-6, LIF, steel factor (Stl) GM-CSF, G-CSF, MIP-1α, and Flk2/Flt3, preferably including Stl. The factors employed may be naturally occurring or synthetic, e.g., prepared recombinantly, and preferably human.

A gene encoding a detectable substance may be integrated into cells for the identification of transformed cells. For example, a gene which encodes a protein such as β-galactosidase, chloramphenicol acetyltransferase, firefly luciferase, or a fluorescent protein marker may be integrated into the cells. Examples of fluorescent protein markers are the Green Fluorescent Protein (GFP) from the jellyfish A. Victoria, or a variant thereof that retains its fluorescent properties when expressed in vertebrate cells. (For example, EGFP commercially available from Clontech Palo Alto, Calif.).

To ensure that the cells have been successfully modified, PCR may be used to amplify vector specific sequences in the transduced cells or their progeny.

Expression of a nucleic acid construct in a modified cell can be controlled in a variety of ways. Thus, the nucleic acid construct may be put under the control of a promoter that will cause the construct or nucleic acid to be expressed constitutively, only under specific physiologic conditions, or in particular cell types. Inducible regulatory elements may be used for gene expression under certain physiologic conditions. By appropriate use of inducible regulatory elements, expression of polypeptide products can be achieved in response to particular stimuli such as chemicals, chemo-attractants, particular ligands, and the like.

The invention further provides a method for preparing a chimeric polypeptide comprising a CD3 agonist and a gastrin compound and optionally an exogenous polypeptide or a GLP-1 agonist utilizing a nucleic acid construct of the invention. In an embodiment a method for preparing a chimeric polypeptide comprising a CD3 agonist and a gastrin compound is provided comprising (a) transferring a vector comprising a CD3 agonist and a gastrin compound into a host cell; (b) selecting transformed host cells from untransformed host cells; (c) culturing a selected transformed host cell under conditions which allow expression of the chimeric polypeptide; and (d) isolating the chimeric polypeptide. The invention still further provides a chimeric polypeptide produced by a method of the invention.

Chimeric polypeptides of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

In embodiments of the invention the host cell is a stem cell, an islet precursor cell, mature insulin secreting cell or pancreatic insulin secreting β cell. Therefore, the invention provides a method for genetically modifying stem cells, islet precursor cells, mature insulin secreting cells or pancreatic insulin secreting β cells with a nucleic acid construct of the invention comprising obtaining stem cells, islet precursor cells, mature insulin secreting cells or pancreatic insulin secreting β cells to be genetically modified, providing the cells ex vivo with conditions for cell proliferation, and genetically modifying the cells with the nucleic acid construct. In an aspect of a method of the invention, a nucleic acid construct comprising a sequence encoding a CD3 agonist and a second nucleic acid comprising a sequence encoding a gastrin compound are employed. In some methods of the invention, a construct may include a sequence encoding a GLP-1 agonist, or a nucleic acid encoding a GLP-1 agonist may also be introduced. The invention contemplates modified islet precursor cells or modified stem cells produced by methods of the invention. A modified cell of the invention may comprise an inducible regulatory element which when activated results in expression of the nucleic acid construct thereby effecting proliferation and/or expansion of the cells.

A nucleic acid construct can provide enhanced expansion of cells including stem cells, islet precursor cells, or mature insulin-secreting cells.

Cell preparations comprising modified islet precursor cells or modified stem cells, or expanded or differentiated cells may be used in both cell therapies (e.g. transplantation) and gene therapies aimed at alleviating a condition and/or disease.

Modified cells in an expanded cell preparation may also be used in cellular gene therapy. According to an aspect of the invention, modified cells in an expanded cell preparation may be transfected with a desired gene that can be used for treatment of genetic diseases. Thus, the cells may be modified to produce a product to correct a genetic deficiency, or where the host has acquired a genetic deficiency through a subsequent disease. For example, cell-related genetic diseases can be treated by grafting the expanded cell preparation with cells transfected with a gene that can make tip for the deficiency or the abnormality of the gene causing the diseases. Further, an expanded preparation comprising normal cells free from abnormalities of genes (from a suitable donor) can be used for treatment.

Modified cells or expanded cell preparations can be introduced in a vertebrate, which is a recipient of cell grafting, by, for example, conventional intravenous administration.

### Compositions, Conjugates and Methods

The invention is related to compositions, nucleic acid constructs, conjugates, and methods that utilize one or more CD3 agonist and a gastrin compound, and optionally a GLP-1 agonist, or sequence encoding same, to provide beneficial effects, in particular enhanced beneficial effects. Embodiments of the invention may provide beneficial effects on the attenuation of islet cell destruction, whether such islet cells are pre-existing in a given subject or whether they are the result of islet neogenesis, and may provide beneficial effects on insulin production. In particular, the beneficial effects of embodiments of the invention are sustained. In aspects of the invention involving a CD3 agonist and a gastrin compound the beneficial ceffects can be additive or synergistic.

In an embodiment, where the condition or disease is diabetes, sustained beneficial effects of a composition, conjugate, treatment, or combination treatment of the invention can manifest as one or more of the following:
a) An increase in pancreatic insulin levels relative to the levels measured in the absence of a CD3 agonist and a gastrin compound and optionally a GLP-1 agonist, after administration to a subject with symptoms of diabetes. Preferably the compounds induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in pancreatic insulin levels in a subject.
b) A reduction or an absence of symptoms of islet inflammation after administration to a subject with symptoms of diabetes.
c) A decrease in blood glucose levels relative to the levels measured in the absence of a CD3 agonist and a gastrin compound, and optionally a GLP-1 agonist, in subjects with symptoms of diabetes. Preferably, the compound(s) induce at least about a 2%, 5%, 10%, 15%, 20%, 30%, 40%, 50%, 60%, 70%, 80%, or 90% decrease in blood glucose levels. Most preferably, the compound(s) yield blood glucose levels about or close to the levels common in a normal subject.
d) An improvement in glucose tolerance. In particular, at least about a 5-95%, 10-90%, 10-80%, 10-70%, 10-60%, improvement in glucose tolerance.
c) An increase in C-peptide levels relative to the levels measured in the absence of a CD3 agonist and a gastrin compound, and optionally a GLP-1 agonist, in subjects with symptoms of diabetes. Preferably, the compounds induce at least about a 0.05%, 0.1%, 0.5%, 1%, 2%, 5%, 10%, 15%, 20%, 30%, 33%, 35%, 40%, 45%, 50%, 60%, 70%, 80%, 90%, 95%, or 99% increase in C-peptide levels.
f) Maintenance of blood glucose levels at about normal for a prolonged period of time, in particular for at least 1 week, 2 weeks, 3 weeks, 4 weeks, 5 weeks, 6 weeks, 8 weeks, 10 weeks, 12 weeks, 14 weeks, 16 weeks, 20 weeks, 24 weeks, 30 weeks, 40 weeks, 52 weeks, 78 weeks, or several years, more particularly, 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, or 2 weeks to 18 months.
g) A reduction, prevention, or slowing of the rate of disease progression in a subject with diabetes.
h) A reduction or prevention of the development of severe hyperglycemia and ketoacidosis with symptoms of diabetes.
i) An increase of beta cell function, in particular an increase in beta cell function sustained over 4 weeks, 5 weeks, 6 weeks, 8 weeks, 10 weeks, 12 weeks, 14 weeks, 16 weeks, 20 weeks, 24 weeks, 30 weeks, 40 weeks, 48 weeks, 52 weeks, or 78 weeks, or several years.
j) An improved prognosis with respect to microvascular and macrovascular complications.
k) An increase in survival in a subject with symptoms of diabetes.
l) A decrease in requirement for insulin injection/intake by at least 10-90%, 10-80%, 10-70%, 10-60%, 10-50%, 10-40%, 10-30%, or 10-20%.

One or more of these beneficial effects can be demonstrated in a diabetic subject or disease model, for example, a non-obese (NOD) mouse with symptoms of diabetes.

A CD3 agonist, a gastrin compound, and optionally a GLP-1 agonist may be selected for particular applications in the present invention based on one or more of the following characteristics: ability to initiate a signal transduction pathway resulting in proliferation and/or differentiation of beta cells or insulinotropic activity; increased beta cell function; ability to reduce glucose levels, insulinotropic activity; stimulation of beta cell proliferation/differentiation; and/or, an in vivo half-life of at least about 5 minutes to 24 hours, preferably 2 to 10 hours or 2 to 8 hours in humans using conventional methods.

In aspects of the invention, the CD3 agonist is an anti-CD3 antibody or F(ab′)₂ fragment thereof, preferably a humanized anti-CD3 antibody or F(ab′)₂ fragment thereof. In particular aspects, the CD3 agonist is monoclonal antibody OKT3, hOKT3-γl (Ala-Ala), CD3 mAb 145 2Cl 1, YTH 12.5.14.2, YTH 12.5, CAMPATH, or a F(ab′)₂ fragment of any of the foregoing.

In other aspects of the invention, the gastrin compound is gastrin 17 and analogs and derivatives thereof. In a particular embodiment, the gastrin compound is synthetic human gastrin I having 17 amino acid residues with a Leu residue at amino acid position 15.

In still further aspects, a GLP-1 agonist is exendin-4 or a GLP-1(7-37), GLP-1(7-36) amide, or an analog or derivative of any of the foregoing. In particular, the GLP-1 agonist is a stable GLP-1 analog/derivative, in particular a derivative of GLP-1(7-36) amide or GLP-1(7-37) which comprises a lipophilic substituent. In an embodiment, the GLP-1 agonist is Arg⁴, Lys²⁶(N^{ε}-(γ-Glu(N^{α}-hexadecanoyl)))-GLP-1(7-37).

The invention contemplates a composition, preferably a pharmaceutical composition, comprising a CD3 agonist and a gastrin compound, and optionally a GLP-1 agonist. In an aspect, the composition provides beneficial effects relative to each compound alone. In another aspect, the invention contemplates a pharmaceutical composition comprising a CD3 agonist and a gastrin compound, and optionally a GLP-1 agonist, which provides beneficial effects, preferably sustained beneficial effects, following treatment.

Pharmaceutical compositions of the invention can be selected that have statistically significant sustained beneficial effects, preferably sustained beneficial effects, compared with a CD3 agonist or a gastrin compound alone, or a CD3 agonist and a GLP-1 agonist alone. In an embodiment, a pharmaceutical composition with statistically significant sustained beneficial effects is provided comprising a CD3 agonist that is a humanized anti-CD3 antibody.

In an embodiment, a pharmaceutical composition with statistically significant beneficial effects is provided comprising a CD3 agonist which is a humanized anti-CD3 antibody and a gastrin compound selected from the group consisting of gastrin 17 and analogs and derivatives thereof, preferably synthetic human gastrin I having 17 amino acid residues with a Leu residue at amino acid position 15.

In another embodiment, a pharmaceutical composition with statistically significant beneficial effects is provided comprising CD3 monoclonal antibodies and gastrin-17(leu).

In a further embodiment a composition of the invention comprises a CD3 agonist, a gastrin compound and a GLP-1 agonist. In a particular embodiment a composition of the invention comprises an anti-CD3 antibody (e.g., monoclonal OKT3), a gastrin compound (e.g., synthetic human gastrin I having 17 amino acid residues with a Leu residue at amino acid position 15), and a derivative of GLP-1 (7-36) amide or GLP-1 (7-37) which comprises a lipophilic substituent (e.g. Arg³⁴, Lys²⁶(N^{ε}-(γ-Glu(N^{α}-hexadecanoyl))-GLP-1(7-37)).

In a still further embodiment, a pharmaceutical composition is provided which has been adapted for administration to a subject to provide sustained beneficial effects to treat a condition or disease, in particular diabetes, more particularly Type 1 diabetes and LADA. In a preferred embodiment, it is in a form such that administration to a subject results in blood glucose levels that are about normal that persist in the subject for a prolonged period of time after cessation of treatment, or increased and sustained beta cell function.

In a still further embodiment, a composition comprising a CD3 agonist and a gastrin compound have greater sustained insulinotropic activity following treatment compared with the activity of a CD3 agonist or gastrin compound alone or greater than a CD3 agonist and GLP-1 agonist alone.

This invention provides a conjugate comprising a CD3 agonist linked to a gastrin compound wherein the linkage is, for example, via an amino or a carboxyl group. The invention also relates to isolated covalent conjugates of the invention, and compositions comprising covalent conjugates of the invention. A CD3 agonist may be conjugated to a species via an ester bond between an —OH and a —COOH of a gastrin compound.

Conjugates of a CD3 agonist and a gastrin compound may be conjugated or linked with an intermediate spacer or linker. A suitable spacer or linker may be a mono- or disaccharide, an amino acid, a sulfate, a succinate, an acetate, or an oligomeric polymeric spacer or linker comprising one or more of such moieties.

The invention also provides methods of preparing the above covalent conjugates that result in conjugates with improved pharmacokinetic properties, biological activity, and beneficial effects. The methods comprise incubating or reacting the CD3 agonist with the gastrin compound under conditions that allow formation of a covalent linkage between the two compounds.

The invention therefore contemplates a process for preparing a covalent conjugate comprising a CD3 agonist covalently bonded or linked to a gastrin compound, the process comprising: incubating or reacting the CD3 agonist with a gastrin compound under conditions and at a pH and for a time sufficient for formation of a covalent bond or linkage between the CD3 agonist and gastrin compound; and isolating the covalent conjugate.

The above process for preparing a conjugate comprising a CD3 agonist and a gastrin compound can provide a conjugate with a substantial amount of a CD3 agonist covalently linked to the gastrin compound.

A conjugate of the invention may be a chimeric polypeptide comprising a CD3 agonist or portion thereof and a gastrin compound. N-terminal or C-terminal fusion proteins or chimeric polypeptides, comprising a CD3 agonist conjugated with a gastrin compound, optionally with a spacer or linker, may be prepared by fusing, through recombinant techniques, the N-terminal or C-terminal sequence of a CD3 agonist and the sequence of a gastrin compound.

The invention also provides a conjugate or chimeric polypeptide prepared by a process described herein. The invention also relates to pharmaceutical formulations comprising conjugates (e.g. chimeric polypeptides) of the invention and a pharmaceutically acceptable carrier, excipient, or vehicle.

The invention further relates to a pharmaceutical formulation of a substantially pure covalent conjugate or chimeric polypeptide comprising a CD3 agonist covalently linked to a gastrin compound which provides beneficial effects preferably sustained beneficial effects compared to the CD3 agonist alone.

In an embodiment, a pharmaceutical formulation is provided consisting essentially of covalent conjugates comprising a CD3 agonist covalently linked without an intermediate spacer or linker to a gastrin compound. In another embodiment, a pharmaceutical formulation is provided consisting essentially of covalent conjugates comprising a CD3 agonist covalently linked with an intermediate spacer or linker to a gastrin compound.

The invention also provide a multispecific antibody comprising an antibody conjugate containing anti-CD3 antibodies or fragments thereof or antibody proteins or fragments thereof linked to an antibody or antibody fragment specific for a gastrin compound. A multispecific antibody is an antibody that can bind simultaneously to at least two targets that are of different structure, for example two different antigens. One specificity would be for CD3 and another specificity would be to a gastrin compound. Multispecific, multivalent antibodies are constructs that have more than one binding site, and the binding sites are of different specificity. In aspects of the invention, a bispecific antibody is employed that can bind simultaneously to two targets that are of different structure. Bispecific antibodies (bsAb) and bispecific antibody fragments (bsFab) have at least one arm that specifically binds to, for example, CD3 and at least one other arm that specifically binds to a gastrin compound. Bispecific fusion proteins can be produced using molecular engineering. In an embodiment, the bispecific fusion protein is monovalent, comprising a scFv with a single binding site for one antigen and a Fab fragment with a single binding site for a second antigen. In another form, the bispecific fusion protein is divalent, consisting of, for example, an IgG with a binding site for one antigen and two scFv with two binding sites for a second antigen. In an embodiment, a bispecific antibody conjugate is provide comprising an antibody that can bind to CD3, an antibody that can bind to a gastrin compound, and a bound gastrin compound.

A pharmaceutical composition or formulation may optionally comprise a pharmaceutically acceptable carrier, excipient, or vehicle as described herein.

### Applications

The invention contemplates the use of a CD3 agonist, and optionally a gastrin compound alone or with a GLP-1 agonist, nucleic acid construct, composition, conjugate, and combination treatment of the invention for preventing, and/or ameliorating disease severity, disease symptoms, and/or periodicity of recurrence of a condition and/or disease. The invention also contemplates treating, in mammals, conditions and/or diseases using a CD3 agonist and optionally a gastrin compound with or without a GLP-1 agonist, nucleic acid construct, composition, conjugate or treatment of the invention. In particular, the present invention provides improved methods and compositions for use of a CD3 agonist and a gastrin compound, and optionally a GLP-1 agonist, for sustained treatment of diabetes, in particular Type 1 diabetes and LADA.

The present invention includes combination treatments providing additive or synergistic activity, delivering an additive or synergistically effective amount, or an amount to provide a therapeutically effective amount of a CD3 agonist and a gastrin compound, or a conjugate or composition of the invention. Pharmaceutical compositions suitable for use in the present invention include compositions wherein the active ingredients are contained in a therapeutically effective amount, in particular a synergistically effective amount.

The present invention in an embodiment provides a composition comprising a CD3 agonist and a gastrin compound, and optionally a GLP-1 agonist that achieves greater efficacy, potency, and utility. The greater efficacy can be shown by improving glucose tolerance in diabetes with treatment resulting in sustained improvement of blood glucose after ceasing treatment and also in recent onset diabetes. An improvement in glucose tolerance may also be observed with the compositions described herein using lower doses of CD3 agonist, i.e., doses below 1-50 μg/kg body weight, in particular, 1-30 μg/kg body weight. Greater efficacy can also be illustrated by increased beta cell function, in particular sustained increased beta cell function.

Greater efficacy and potency of a treatment of the invention comprising a CD3 agonist and a gastrin compound, and optionally a GLP-1 agonist, may improve the therapeutic ratio of treatment, reducing untoward side effects and toxicity. The methods of the invention also have utility in improving long-standing diabetes even when treatment is begun long after the completion of β cell destruction.

The invention also relates to a method of treatment comprising administering a therapeutically effective amount of at least one CD3 agonist in combination with the administration of at least one gastrin compound, and optionally a GLP-1 agonist, which upon administration to a subject with symptoms of diabetes produces beneficial effects, preferably sustained beneficial effects, manifested as reduced blood glucose levels, increased beta cell function, and/or increased pancreatic insulin.

In an aspect, the CD3 agonist is administered in a regimen, which additionally comprises administration of a gastrin compound and optionally a GLP-1 agonist.

In another aspect, the CD3 agonist and a gastrin compound are administered in suboptimal dosages i.e. dosages lower than the optimal dosages for single compound therapy.

In a further aspect, the CD3 agonist and a gastrin compound are administered in therapeutically effective amounts and for a sufficient time to produce a beneficial effect.

In a further aspect, the CD3 agonist and a gastrin compound are administered in therapeutically effective amounts and for a sufficient time to produce a synergistic effect.

In an aspect of the invention therapeutically effective amounts of a CD3 agonist and a gastrin compound, and optionally a GLP-1 compound, are combined prior to administration to a subject. In an embodiment, therapeutically effective amounts of a CD3 agonist and a gastrin compound, and optionally a GLP-1 compound, are mixed at a physiologically acceptable pH.

In a further embodiment, the invention provides a method for preventing and/or treating Type 1 or Type 2 diabetes comprising administering a therapeutically effective amount of a composition or conjugate of the invention, or administering in combination a CD3 agonist and a gastrin compound, and optionally a GLP-1 compound.

In a further embodiment, the invention provides a method for ameliorating progression of disease or obtaining a less severe stage of disease in a person suffering from Type 1 or Type 2 diabetes comprising administering a therapeutically effective amount of a composition or conjugate of the invention, or administering in combination a CD3 agonist and a gastrin compound, and optionally a GLP-1 compound.

The invention relates to a method of delaying the progression of impaired glucose tolerance or non-insulin requiring Type 2 diabetes to insulin requiring Type 2 diabetes comprising administering a therapeutically effective amount of a composition or conjugate of the invention, or administering in combination a CD3 agonist and a gastrin compound.

The invention relates to a method of improving beta cell function in a subject with Type 1 diabetes or LADA comprising administering a therapeutically effective amount of a composition or conjugate of the invention, or administering in combination a CD3 agonist and a gastrin compound, and optionally a GP-1 compound.

The invention also relates to a method of increasing the insulin synthesis capability of a subject comprising administering a therapeutically effective amount of a composition or conjugate of the invention, or administering in combination a CD3 agonist and a gastrin compound, and optionally a GLP-1 agonist.

Prolonged efficacious islet cell neogenesis can be achieved in accordance with the invention following administration of a CD3 agonist and a gastrin compound, and optionally a GLP-1 agonist, a nucleic acid construct, or a composition or conjugate of the invention. The CD3 agonist, a gastrin compound, and optionally a GLP-1 agonist, or construct, composition, or conjugate can be administered in vivo to provide for proliferation and/or differentiation of islet cells in a subject or it can be administered ex vivo to cells for transplantation. A CD3 agonist, gastrin compound, and optionally GLP-1 agonist, or composition, construct, or conjugate can be introduced to cells using methods known to a person skilled in the art including recombinant techniques. For example, a chimeric insulin promoter-CD3 agonist fusion gene may be introduced in vivo or ex vivo to pancreatic cells to express one or more CD3 agonist.

The invention relates to a method for differentiating stem cells or progenitor cells into insulin secreting cells comprising contacting the stem cells or progenitor cells with a therapeutically effective amount of a CD3 agonist and a gastrin compound, and optionally a GLP-1 agonist, or a nucleic acid construct, composition, or conjugate of the invention or sufficient amounts of a CD3 agonist and gastrin compound and optionally GLP-1 agonist, or a nucleic acid construct, composition or conjugate to differentiate stem cells or progenitor cells. The stem cells may be obtained from pancreatic islets, umbilical cords, embryos, or stem cell lines. The amount and duration of differentiation is significantly different compared with that achieved in the absence of the CD3 agonist, gastrin compound and optionally GLP-1 agonist, or a composition, or conjugate of the invention. In an embodiment, the stem cells or progenitor cells are contacted with the CD3 agonist and gastrin compound, and optionally a GLP-1 agonist, nucleic acid construct, a composition or conjugate of the invention in culture. In another embodiment, the stem cells or progenitor cells are contacted with the CD3 agonists and gastrin compound and optionally GLP-1 agonist, construct, or composition or conjugate in a subject. The CD3 agonists and gastrin compounds and optionally GLP-1 agonists, constructs, compositions or conjugates, may be administered to a subject before, during, or after implantation of stem cells in the subject to expand and differentiate the stem cells in the subject for a prolonged period. The method may additionally comprise administering an immunosuppressive agent.

In an aspect, the invention provides a method for treating diabetes mellitus by providing a composition comprising a CD3 agonist and a gastrin compound and optionally a GLP-1 agonist in an amount sufficient to effect differentiation of pancreatic islet precursor cells to mature insulin-secreting cells for a prolonged period following administration. A CD3 agonist and gastrin compound and optionally a GLP-1 agonist can be administered systemically, in particular by injection, preferably intravenously, in a physiologically acceptable carrier. Alternatively, a CD3 agonist and gastrin compound and optionally a GLP-1 agonist can be expressed in situ, and pancreatic islet precursor cells are transformed either ex vivo or in vivo with one or more nucleic acid encoding a CD3 agonist and a gastrin compound and optionally a GLP-1 agonist in an expression construct vector that provides for expression of the compounds in the cells. The expression construct can include regulatory elements.

The invention also relates to inducing islet neogenesis in a subject comprising contacting islet precursor cells with a CD3 agonist and a gastrin compound and optionally a GLP-1 agonist, or a nucleic acid construct, composition or conjugate of the invention in a sufficient amount to increase and prolong proliferation of islet precursor cells in the subject thereby inducing islet neogenesis. In an aspect, the invention provides a method for stimulating prolonged beta cell proliferation in a subject comprising administering a therapeutically effective amount of a CD3 agonist, a gastrin compound and optionally a GLP-1 agonist, or a nucleic acid construct, composition, or conjugate of the invention. In an embodiment, the invention provides a method for increasing the number size, and/or functionality of beta cells in a subject for a prolonged period comprising administering a therapeutically effective amount of a CD3 agonist, a gastrin compound and optionally a GLP-1 agonist, or a nucleic acid construct, composition, or conjugate of the invention.

Regenerative differentiation of pluripotent pancreatic precursor cells, for example, pancreatic ductal cells, into mature insulin-secreting cells for a prolonged period can be obtained with one or more CD3 agonist, gastrin compound and optionally a GLP-1 agonist, nucleic acid constructs, conjugates, compositions and methods described herein for treatment of diabetes mellitus, particularly juvenile onset diabetes, Type 1 diabetes, and LADA, and by therapeutic administration of one or more CD3 agonist and a gastrin compound and optionally a GLP-1 agonist, nucleic acid constructs, conjugates, or compositions of the invention which are provided for systemic administration, or for in situ expression within the pancreas.

The invention provides methods for treating diabetes mellitus in a patient in need thereof by administering a composition comprising a CD3 agonist, a gastrin compound and optionally a GLP-1 agonist, or a composition or conjugate of the invention, in an amount sufficient to effect prolonged differentiation of the patient's pancreatic islet precursor cells to mature insulin-secreting cells and/or to stimulate insulin synthesis in existing islet cells. The agonist, compounds, composition, etc can be administered systemically or expressed in situ by host cells containing a nucleic acid construct in an expression vector wherein the nucleic acid construct comprises a coding sequence for a CD3 agonist and for a gastrin compound, or optionally a GLP-1 agonist, or a chimeric polypeptide together with transcriptional and translational regulatory elements functional in pancreatic islet precursor cells.

In an aspect, the invention provides a method for treating diabetes mellitus in a patient in need thereof which includes administering to the individual a CD3 agonist, a gastrin compound, and optionally a GLP-1 agonist, or a nucleic acid construct, composition, or conjugate of the invention in a dose sufficient to effect prolonged differentiation of pancreatic islet precursor cells to mature insulin-secreting cells. In another aspect, the invention provides a method for treating insulin dependent diabetes, especially Type 1 or juvenile diabetes mellitus or LADA, comprising administering, preferably systemically, a differentiation regenerative amount of a CD3 agonist, a gastrin compound, and optionally a GLP-1 agonist, or a construct, composition, or conjugate of the invention to a diabetic mammal, to stimulate islet neogenesis resulting in an increase in the number of functional glucose responsive insulin secreting β cells in the pancreas for a prolonged period following administration.

The invention contemplates a method of expanding a functional beta cell mass of pancreatic islet transplants in a diabetic patient for a prolonged period, the method comprising administering to the patient a therapeutically effective amount of a CD3 agonist, a gastrin compound, and optionally a GLP-1 agonist, or a nucleic acid construct, composition, or conjugate of the invention.

The invention in an embodiment provides a method for preventing and/or treating diabetes, the method comprising administering to a mammal in need thereof a CD3 agonist, a gastrin compound, and optionally a GLP-1 agonist, or a nucleic acid construct, composition, or conjugate of the invention in an amount sufficient to increase the number of pancreatic insulin secreting β cells in the mammal for a prolonged period following administration, thereby preventing and/or treating the diabetes. The composition is administered systemically. The mammal is a diabetic mammal, for example, the mammal has been diabetic for an extent of 1% of the lifespan of the mammal. The CD3 agonist, gastrin compound, and optionally GLP-1 agonist, or construct, composition, or conjugate is provided in an amount sufficient to induce differentiation of the pancreatic islet precursor cells into glucose responsive insulin secreting islet cells for a prolonged period.

Another embodiment of the invention provides a method for preventing and/or treating diabetes, the method comprising administering to a mammal in need thereof a CD3 agonist, a gastrin compound, optionally a GLP-1 agonist, or a nucleic acid construct, a composition, or conjugate of the invention in an amount sufficient to increase the amount and duration of proliferation of islet precursor cells in pancreatic tissue for a prolonged period following administration, thereby preventing and/or treating the diabetes.

In another aspect, the invention provides a method for preventing and/or treating diabetes, the method comprising administering to a mammal in need thereof a CD3 agonist, a gastrin compound, and optionally a GLP-1 agonist, or a nucleic acid construct, composition, or conjugate of the invention, in an amount sufficient to increase the number of pancreatic insulin secreting β cells in the mammal for a prolonged period following administration; and determining the amount of islet neogenesis, thereby preventing and/or treating the diabetes. The amount of islet neogenesis may be measured by one or more of the following parameters: blood glucose, serum glucose, blood glycosylated hemoglobin, pancreatic β cell mass, beta cell function serum insulin, and pancreatic insulin content. Administering the composition can reduce blood glucose compared to blood glucose assayed prior to administering the composition. Glycosylated hemoglobin concentration can be reduced for a prolonged period compared to glycosylated hemoglobin concentration in the mammal assayed prior to administering the compounds, constructs, conjugates or compositions. Serum insulin concentration can be increased for a prolonged period compared to serum insulin concentration in the mammal assayed prior to administering the composition. Pancreatic insulin concentration can be increased for a prolonged period compared to pancreatic insulin concentration in the mammal assayed prior to administering the composition.

In a further aspect, the invention provides a method for inducing pancreatic islet neogenesis in a mammal, the method comprising administering to the mammal a CD3 agonist, a gastrin compound, and optionally a GLP-1 agonist, or a composition, or conjugate of the invention, in an amount sufficient to increase the amount and duration of proliferation of islet precursor cells in pancreatic tissue for a prolonged period following administration, thereby inducing pancreatic islet neogenesis. The plurality of cells can be multicellular. The plurality of cells is delivered systemically to the mammal.

In a still further aspect, the invention provides a method for inducing islet neogenesis therapy in a cell of an animal for a prolonged period, comprising contacting the cell with a nucleic acid sequence encoding a CD3 agonist operably linked to a regulatory element, for example, an insulin promoter receptor ligand, for example, a metallothionein promoter, and a nucleic acid sequence encoding a gastrin compound, and optionally a nucleic acid sequence encoding a GLP-1 agonist. For example, the cell is a germ cell, or the cell is an autologous cell cultured ex vivo.

The invention contemplates cell based treatment methods using a CD3 agonist, a gastrin compound, and optionally a GLP-1 agonist, or nucleic acid construct, composition, or conjugate of the invention. Thus, the invention contemplates methods comprising treating cells, or treating explanted pancreatic tissue of a mammal with a CD3 agonist, a gastrin compound, and optionally a GLP-1 agonist, or a nucleic acid construct, composition, or conjugate of the invention and introducing the treated cells or pancreatic tissue to the mammal to provide beneficial effects, in particular sustained beneficial effects. See PCT/CA03/33595 for a description of general culture and cell based treatment methods.

A method for treating a subject with a condition and/or disease described herein comprises contacting ex vivo a plurality of cells with a CD3 agonist, a gastrin compound, and optionally a GLP-1 agonist, or a nucleic acid construct, composition, or conjugate of the invention, optionally culturing the cells, and administering the cells to the subject in need thereof to provide beneficial effects, in particular sustained beneficial effects.

In embodiments of cell based therapeutic methods the cells are pancreatic ductal cells and the amount of compounds or compositions used in the methods are generally effective to increase the amount of insulin secreting cells in the subject for a prolonged period. The cells may be autologous (i.e. from the same subject), or may be from another individual of the same species, or from a different species.

The invention provides a method of islet cell transplantation comprising obtained stem cells or pancreatic islet precursor cells to be transplanted from a donor; modifying the cells with a nucleic acid construct of the invention, CD3 agonist and gastrin compound, and optionally a GLP-1 agonist, a composition or conjugate of the invention, culturing the cells under proliferation conditions to thereby expand the cells; and transplanting the cells to a patient. In an embodiment, the donor and patient is a single individual.

A gene therapy aspect of the invention comprises removing stem cells or pancreatic islet precursor cells from a subject, transducing the cells in vitro with a nucleic acid construct of the invention comprising an exogenous gene, and administering transduced cells to a subject.

Another gene therapy aspect of the invention comprises removing stem cells or pancreatic islet precursor cells from a subject, transducing the cells in vitro with a nucleic acid construct of the invention comprising an exogenous gene that encodes a therapeutic and administering transduced cells to a subject. The modified cells and their progeny will express the therapeutic in vivo and can repopulate the host system thus providing a sustained therapeutic benefit.

The invention also contemplates a method for treating diabetes in a subject comprising transplanting a pancreatic islet preparation into the subject and administering a therapeutically effective amount of a CD3 agonist, a gastrin compound, and optionally a GLP-1 agonist, or a composition or conjugate of the invention to provide beneficial effects, in particular sustained beneficial effects.

The invention also relates to a method for sustaining islet cells or precursor cells in culture comprising culturing the cells in the presence of a CD3 agonist, a gastrin compound, and optionally a GLP-1 agonist, or a composition or conjugate of the invention in an amount sufficient to sustain the cells in culture. The cells may be sustained in culture for a significantly longer period of time compared with cells cultured in the absence of the compounds, composition, or conjugate. Culturing cells in the presence of a CD3 agonist, a gastrin compound, and optionally a GLP-1 agonist, or a composition or conjugate of the invention will be particularly useful in preparing and maintaining cells intended for transplantation.

Also provided are methods and compositions for treating diabetes in a patient in need thereof by implanting into a diabetic patient pancreatic islet cells that have been exposed in culture to a sufficient amount of a CD3 agonist and a gastrin compound, and optionally a GLP-1 agonist, or a nucleic acid construct, composition, or conjugate of the invention to increase the number of pancreatic beta cells in the islets for a prolonged period; optionally the population of pancreatic beta cells can be grown in culture for a time sufficient to expand the population of β-cells prior to transplantation.

Another embodiment of the invention provides a method for treating diabetes, the method comprising: contacting ex vivo a plurality of cells with a composition comprising a CD3 agonist, a gastrin compound, and optionally a GLP-1 agonist, or a nucleic acid construct, a composition, or conjugate of the invention in an amount sufficient to increase proliferation of islet precursor cells and the amount of insulin secreting islet cells, and administering the contacted plurality of cells to a mammal in need thereof to produce a beneficial effect, in particular a sustained beneficial effect. The cells can be autologous. The composition is provided in an amount sufficient to effect differentiation of stem cells, for example, to effect differentiation of pancreatic islet precursor cells in pancreatic tissue into mature insulin secreting islet cells. The agonists, compound, construct, composition, or conjugate is provided in an amount sufficient to increase proliferation of pancreatic islet stem cells, for example, of pancreatic islet precursor cells for a prolonged period. Stem cells can be obtained either from a pancreatic tissue or from a non-pancreatic tissue, such as liver or bone marrow.

The invention provides a method of treating a condition and/or disease comprising administering a CD3 agonist and a gastrin compound, and optionally a GLP-1 agonist, or a nucleic acid construct, composition, or conjugate of the invention with a plurality of cells to a subject in need thereof to thereby produce a beneficial effect, preferably a sustained beneficial effect. In an aspect, the invention provides a method for expanding and differentiating stem cells, in a diabetic recipient of the cells, into insulin secreting cells, the method comprising implanting the cells in the recipient, and administering a composition containing an effective dose of a CD3 agonist, a gastrin compound, and optionally a GLP-1 agonist, or a composition, nucleic acid construct, or conjugate of the invention to produce a beneficial effect, in particular a sustained beneficial effect. For example, the implanted cells are obtained from a human, for example, are obtained from human pancreatic islets, human liver, human bone marrow, human umbilical cord, or human embryos. Implanting the cells into the recipient may be by a route such as injecting directly into an organ, for example, into the pancreas, the kidney, or the liver. Alternatively, implanting the cells may be administering by intravenous injection, for example, into the portal vein or into the hepatic vein. In certain embodiments, prior to implanting the cells are treated ex vivo with a composition comprising a CD3 agonist and a gastrin compound, and optionally a GLP-1 agonist.

The invention also contemplates the use of a composition comprising at least one CD3 agonist and a gastrin compound, and optionally a GLP-1 agonist, for the preparation of a medicament providing beneficial effects, preferably sustained beneficial effects in treating a condition and/or disease.

In an embodiment, the invention relates to the use of a therapeutically effective amount of at least one CD3 agonist and at least one gastrin compound, and optionally a GLP-1 agonist, for preparation of a medicament for providing beneficial effects, preferably sustained beneficial effects, in treating a condition and/or disease.

In an embodiment the invention provides the use of a CD3 agonist and a gastrin compound, and optionally a GLP-1 agonist, for the preparation of a medicament to increase (preferably prolonged increase) the number, size and/or functionality of beta cells in a subject after treatment.

In another embodiment the invention provides the use of a CD3 agonist and a gastrin compound, and optionally a GLP-1 agonist, for the preparation of a medicament for stimulation (preferably prolonged stimulation) of beta cell proliferation after treatment.

In a still further embodiment the invention provides the use of a CD3 agonist and a gastrin compound, and optionally a GLP-1 agonist, for the preparation of a medicament for prolonged or sustained treatment of Type 1 or Type 2 diabetes, in particular Type 1 diabetes and LADA.

The invention additionally provides uses of a CD3 agonist and a gastrin compound and optionally a GLP-1 agonist, in the preparation of a medicament for beneficial effects, preferably sustained beneficial effects, in the treatment of conditions and/or diseases disclosed herein.

The present invention also includes methods in combination with one or more additional therapeutic agents including without limitation immunosuppressive agents (e.g. rapamycin, cyclosporine, ISAtx247, and FK506), antiobesity agents, antidiabetic agents, appetite regulating drugs, antihypertensive agents, agents for the treatment and/or prevention of complications resulting from or associated with a condition and/or disease, in particular diabetes and obesity, anti-nausea, anti-headache medications, and general medications that treat or prevent side effects. In particular, when the treatment is used in a patient with diagnosed Type 1 or LADA diabetes, co-therapy with insulin, insulin analogues or oral antidiabetic agents will be common. Examples of pharmacologically active substances are: insulin, GLP-1 agonists, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as anti-hyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine; proton pump inhibitors such as omeprazole, esomeprazole, lansoprazole, pantoprazole, rabeprazole sodium; peroxisome proliferator-activated receptor (PPAR) agonists such as azelaoyl PAF, 2-bromohexadecanoic acid, ciglitizone, clofibrate, fenofibrate, Fmo-Leu-Oh, leukotriene B4, prostaglandin A₂, prostaglandin J₂, Rivoglitazone™, Galida™, Muraglitazar™, Naveglitazar™ (LY-818), Netoglitazone™, tetradecyl thioacetic acid, troglitazone, pirinixic acid, tesaglitazar; β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (conicotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptine, doprexin), lipase/amylase inhibitors, RXR (retinoid X receptor) modulators, TR β agonists; histamine H3 antagonists.

Any suitable combination of the compounds according to the invention and optionally one or more further pharmacologically active substances are considered to be within the scope of the present invention.

In aspects of the invention, patients receive state-of-the art therapy with insulin and/or insulin analogs simultaneously during the treatment period in order to provide glycemic control.

Therapeutic efficacy and toxicity of compositions and methods or the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

The methods of the invention may further comprise measuring one or more of the following markers: blood glucose, serum glucose, blood glycosylated haemoglobin, pancreatic beta cell mass, beta cell function, serum insulin, pancreatic insulin levels, morphometrically determined beta cell mass, amount of insulin secreting cells, and glucose responsiveness of insulin secreting cells.

The invention further relates to the use of modified islet precursor and stem cells and preparations comprising same, including expanded cell preparations, in drug discovery. Modified cells described herein can be used to screen for test substances that effect proliferation or differentiation of cells. Thus, the invention provides a method for screening a test substance for its potential to effect proliferation or differentiation of islet precursor cells comprising:
(a) culturing the modified cells comprising a nucleic acid construct or vector of the invention in the presence of the test substance;
(b) detecting the presence or absence of an effect of the test substance on expansion or differentiation of the modified cells whereby an alteration in the amount of expansion or differentiation indicates the test substance effects proliferation or differentiation of the cells.

Still another aspect of the present invention provides a method of conducting a drug discovery business comprising:
(a) providing one or more systems for identifying agents by their ability to inhibit or potentiate expansion or differentiation of modified cells of the invention;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Modified cells and expanded cell preparations of the invention can be used in various bioassays. In an embodiment, modified cells are used to determine biological factors required for proliferation and/or differentiation. Different biological compounds (e.g. hormones, specific growth factors, etc.) can be added in a stepwise fashion to modified cells or expanded cell preparations to identify biological compounds that induce or inhibit proliferation or differentiation of the cells. Other uses in a bioassay for the cells are differential display (i.e. mRNA differential display) and protein-protein interactions using proteins from the cells. Protein-protein interactions can be determined with techniques such as a yeast two-hybrid system. Proteins from modified cells and expanded cell preparations can be used to identify unknown proteins that interact with the cells including but not limited to growth factors, hormones, enzymes, transcription factors, translational factors, and tumor suppressors. Bioassays involving modified cells and expanded cell preparations of the invention, and the protein-protein interactions these cells form and the effects of protein-protein or cell-cell contact may be used to determine how surrounding tissue contribute to proliferation of the cells.

In an aspect, the invention provides a culture system comprising modified islet precursor or stem cells from which genes, proteins, and other metabolites involved in proliferation of the cells can be identified and isolated. The cells in a culture system of the invention may be compared with other cells (e.g. differentiated cells) to determine the mechanisms and compounds that stimulate production of the cells.

The invention also provides a transgenic animal whose germ cells comprise a nucleic acid sequence encoding a mammalian CD3 agonist operably linked to a regulatory element (e.g. heterologous promoter) and a nucleic acid sequence encoding a mammalian gastrin-compound operably linked to a regulatory element (e.g. heterologous promoter).

### Administration and Formulations

A CD3 agonist, a gastrin compound, and optionally a GLP-1 agonist, a composition, nucleic acid construct, or conjugate of the present invention can be administered by any means that produce contact of the active agent(s) with the agent's site of action in the body of a subject or patient to produce a beneficial effect, in particular a sustained beneficial effect. The active ingredients can be administered simultaneously or sequentially and in any order at different points in time, to provide the desired beneficial effects, in particular sustained beneficial effects. A CD3 agonist and a gastrin-compound and optionally a GLP-1 agonist, a composition, or conjugate of the invention can be formulated for sustained release, for delivery locally or systemically. It lies within the capability of a skilled physician or veterinarian to select a form and route of administration that optimizes the effects of the compositions and treatments of the present invention to provide beneficial effects, in particular sustained beneficial effects.

Modes of parenteral administration include, but are not limited to, transdermal, intramuscular, intraperitoneal, intravenous, subcutaneous, intranasal, and oral routes. The compounds may be administered by any convenient route, for example, by infusion or bolus injection, by absorption through epithelial or mucocutaneous linings (e.g. oral mucosa, rectal and intestinal mucosa, etc.), and may be administered together with other biologically active agents. A preferred route of administration is systemic, for example, by subcutaneous injection. For parenteral administration, the compounds and compositions described herein may be combined with saline, PBS, or other suitable buffer, at an appropriate pH. A sustained release formulation can also be used for either or both therapeutic agents.

The CD3 agonist, a gastrin compound, and optionally a GLP-1 agonist, a composition, or conjugate may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

According to an aspect of the invention, the CD3 agonist and gastrin compound and optionally a GLP-1 agonist are provided in the form of a composition suitable for administration by injection. Such a composition can either be an injectable solution ready for use or it can be an amount of a solid composition, such as a lyophilized product, which has to be dissolved in a solvent before administration.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

An amount of a therapeutic of the invention which will be effective in the treatment of a particular condition or disorder to provide effects, in particular sustained beneficial effects, will depend on the nature of the condition or disorder, and can be determined by standard clinical techniques. The precise dose to be employed in the formulation will also depend on the route of administration, and the seriousness of the condition or disorder, and should be decided according to the judgment of the practitioner and each patient's circumstances. Routine determinations of blood levels of insulin or C peptide, and of fasting levels of glucose or glucose challenges, are determined by one of ordinary skill in the art.

Suitable dosage ranges for administration are particularly selected to provide beneficial effects, in particular sustained beneficial effects. The dosage ranges are generally about 0.01 micrograms to about 500 micrograms of a CD3 agonist or gastrin compound per kilogram body weight per day, for example, about 0.01 micrograms to about 1 micrograms/kg, about 0.1 micrograms/kg to about 10 micrograms/kg, or about 1 microgram/kg to about 50 micrograms/kg.

In another aspect the invention provides a pharmaceutical composition comprising between 0.1 to 20, 0.1 to 30, 0.1 to 40, 0.1 to 50, and 0.1 to 60 micrograms/kg/day CD3 agonist and 0.1 to 20, 0.1 to 30, 0.1 to 40, 0.1 to 50, and 0.1 to 60 micrograms/kg/day gastrin compound.

In particular embodiments of the invention providing sustained beneficial effects, the dosage range for administration of a CD3 agonist or gastrin compound is 1-30 micrograms/kg body weight, in particular 3-30 micrograms/kg body weight, more particularly 5-20 micrograms/kg body weight.

In an embodiment, the composition comprises a CD3 agonist and a gastrin compound in doses that are equal to or at least 1.1, 1.5, 2, 3, 4, 5, 6, 7, 8, 9, or 10 fold lower than the doses of each compound required to provide beneficial effects, preferably sustained beneficial effects, to treat a condition and/or disease.

A composition or treatment of the invention may comprise a unit dosage of at least one CD3 agonist and a unit dosage of at least one gastrin to provide beneficial effects, in particular sustained beneficial effects. A “unit dosage” refers to a unitary dose i.e., a single dose which is capable of being administered to a patient, and which may be readily handled and packed, remaining as a physically and chemically stable unit dose comprising either the active agents as such or a mixture with one or more solid or liquid pharmaceutical excipients, carriers, or vehicles.

In an aspect, a pharmaceutical composition is provided comprising a therapeutically effective suboptimal dosage of a CD3 agonist and a gastrin compound that is effective at decreasing or reducing glucose levels for a sustained period or increasing beta cell proliferation or differentiation following treatment.

In an aspect, an improved pharmaceutical composition is provided comprising therapeutically effective suboptimal amounts of a CD3 agonist and a gastrin compound in a form for chronic or acute therapy of a disease or condition, in particular diabetes.

In an aspect the invention provides a pharmaceutical composition comprising 30-3000, 100-3000, 100-6000, 1000-6000, 2000-6000, and 3000-6000 micrograms each of a CD3 agonist and a gastrin compound per single unit.

In another aspect, the ratio of CD3 agonist to gastrin compound in a composition of the invention is selected to augment the activity of the CD3 agonist and/or gastrin compound and to provide beneficial effects, preferably sustained beneficial effects.

A CD3 agonist and a gastrin compound may be in a ratio selected to augment the activity of one or both compounds to produce beneficial effects, in particular a sustained beneficial effect, and/or to produce an additive or synergistic effect. In embodiments, the ratio of a CD3 agonist to a gastrin compound may be from 1:1 to 1:110, 1:1 to 1:100, 1:1 to 1:75, 1:1 to 1:50, 1:1 to 1:25, 1:1 to 1:10, 1:1 to 1:5, and 1:1. In other particular embodiments, the ratio of a gastrin compound to a CD3 agonist may be from 1:1 to 1:110, 1:1 to 1:100, 1:1 to 1:75, 1:1 to 1:50, 1:1 to 1:25, 1:1 to 1:10, and 1:1 to 1:5.

A CD3 agonist may be used in combination with a gastrin compound at therapeutically effective weight ratios of between about 1:1 to 1:150, in particular 1:1 to 1:50. In another embodiment, a gastrin compound may be used in combination with a CD3 agonist at therapeutically effective weight ratios of between about 1:1 to 1:150, in particular 1:1 to 1:50.

A composition or formulation of the invention may be administered to a subject for about or at least about 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, or 2 weeks to 18 months, 2 weeks to 24 months, or several years, periodically or continuously. A composition of the invention may be administered one or more times per day, in particular 1 or 2 times per day.

The compositions of the present invention or fractions thereof typically comprise suitable pharmaceutically acceptable carriers, excipients, and vehicles selected based on the intended form of administration, and consistent with conventional pharmaceutical practices.

Suitable pharmaceutical carriers, excipients, and vehicles are described in the standard text, Remington's Pharmaceutical Sciences, Mack Publishing Company. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

The composition can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

Formulations for parenteral administration of a composition of the invention may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

In an embodiment, a composition herein is formulated in accordance with routine procedures as a pharmaceutical composition adapted for subcutaneous or intravenous administration to human beings to provide a beneficial effect, in particular a sustained beneficial effect. Typically, compositions for subcutaneous or intravenous administration are solutions in sterile isotonic aqueous buffer. Where necessary, the composition may also include a solubilizing agent and a local anesthetic to ameliorate pain at the site of the injection. Generally, the ingredients are supplied either separately or mixed together in unit-dosage form, for example, as a dry, lyophilized powder or water-free concentrate in a hermetically sealed container such as an ampoule or sachette, for example, indicating the quantity of active agent. Where the composition is to be administered by infusion, an ampoule of sterile water or saline for injection can be provided so that the ingredients may be mixed prior to administration.

Compositions of the invention can be formulated as neutral or salt forms. Pharmaceutically acceptable salts include those formed with free amino groups such as those derived from hydrochloric, phosphoric, acetic, oxalic, tartaric acids etc., and those formed with free carboxyl groups such as those derived from sodium, potassium, ammonium, calcium, ferric hydroxides, isopropylamine, triethylamine, 2-ethylamino ethanol, histidine, procaine, etc.

A composition of the invention may be sterilized by, for example, filtration through a bacteria retaining filter, addition of sterilizing agents to the composition, irradiation of the composition, or heating the composition. Alternatively, the compounds or compositions of the present invention may be provided as sterile solid preparations e.g. lyophilized powder, which are readily dissolved in sterile solvent immediately prior to use.

In addition to the formulations described herein, the compositions can also be formulated as a depot preparation. Such long acting formulations may be administered by implantation (for example, subcutaneously or intramuscularly) or by intramuscular injection. Thus, for example, the fractions may be formulated with suitable polymeric or hydrophobic materials (for example, as an emulsion in an acceptable oil), or ion exchange resins, or as sparingly soluble derivatives, for example, as a sparingly soluble salt.

The compositions of the invention and components thereof may comprise soluble polymers as targetable drug carriers.

After pharmaceutical compositions have been prepared, they can be placed in an appropriate container and labeled for treatment of an indicated condition. For administration of a composition of the invention, such labeling would include amount, frequency, and method of administration.

According to another aspect of the invention, a kit is provided comprising a CD3 agonist, a gastrin compound, and optionally a GLP-1 agonist, a composition, conjugate, or nucleic acid construct of the invention. The kit is a package which houses a container which contains a CD3 agonist, a gastrin compound, and optionally a GLP-1 agonist, a composition, nucleic acid construct, or conjugate composition of the invention and also houses instructions for administering to a subject. A kit may contain a single dosage form or it may contain two dosage forms i.e. one for each compound to be administered. In an aspect, the kit comprises a fixed ratio dosage of a CD3 agonist and a gastrin compound.

In embodiments of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the manufacture, use or sale of pharmaceuticals or biological products, which notice reflects approval by the agency of manufacture, use, or sale for human administration.

The invention will be described in greater detail by way of a specific example. The following example is offered for illustrative purposes, and is not intended to limit the invention in any manner. Those of skill in the art will readily recognize a variety of noncritical parameters which can be changed or modified to yield essentially the same results.

### EXAMPLES

### Example 1

The synergistic effects of the combined use of a modulator of CD3 and a gastrin compound in Type 1 diabetes can be measured as follows:

### Study Design and Treatment Protocols:

Upon enrollment and following informed consent, type 1 diabetes patients are randomized into one of four groups: one receiving anti-CD3 and placebo for a gastrin compound, one receiving placebo for anti-CD3 and a gastrin compound, one receiving both anti-CD3 and a gastrin compound and one receiving placebo for both anti-CD3 and a gastrin compound. Anti-CD3 treatment or placebo is administered using a regimen as described by Herold et. al. N Engl J Med 346:1692-98, 2002. Commencing on the same day as the anti-CD3 treatment, the gastrin compound is administered 4 times daily for a period of 3 months (i.e. treatment period). Patients receive state-of-the art therapy with insulin and/or insulin analogs simultaneously during the treatment period in order to provide glycemic control.

### Endpoints:

The primary endpoint is the area under the curve for insulin secretion rates quantified by deconvolution of C-peptide concentrations for the meal test performed after the treatment period. Secondary endpoints include fasting C-peptide, insulin secretion rates after the oral glucose tolerance test, use of exogenous insulin, and HbA 1c. The statistical analysis will be based on baseline subtracted data.

### Baseline Assessment and Data Collection

At baseline, fasting C-peptide is determined, an oral glucose tolerance test and a meal tolerance test are performed, and islet cell antibodies are assessed. HbA 1c, fasting C-peptide, oral glucose tolerance tests and meal tests are repeated between one and seven days following the end of the treatment period, and every three months thereafter for an indefinite period.

### Statistical Analysis

The statistical analysis shows a synergistic effect on the primary endpoint, i.e. the effect of combining anti-CD3 and the gastrin compound is greater than the additive effect of either treatment regimen alone. If the effect of administering placebo for anti-CD3 and placebo for the gastrin compound is designated A, the effect of administering anti-CD3 and placebo for the gastrin compound is designated B, the effect of administering placebo for anti-CD3 and the gastrin compound is designated C and the effect of administering anti-CD3 and the gastrin compound is designated D, then the statistical analysis shows that D-A is greater than (B−A)+(C−A) with statistical significance at the 0.05 level. The statistical test used is a two-way analysis of variance with anti-CD3 and the gastrin compound as the two factors. The interaction term is used to ascertain the presence of synergy.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention. addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

TABLE 1

| GLP-1 agonist | Source |
|---|---|
| DAC:GLP-1 | Conjuchem |
| Long-lasting synthetic glucagons-like peptide | Conjuchem |
| Long-lasting insulinotropic peptides | Conjuchem |
| AC2592 | Amylin Pharmaceuticals/Restoragen |
| AC2993 - Exenatide | Amylin Pharmaceuticals |
| Exendin-4 | Eli Lilly, Alkermes, Amylin |
| NN2211 - GLP-1 (Liraglutide) | Novo Nordisk |
| ThGLP-1 | Theratechnologies |
| ZP10 | Zealand Pharma/Aventis |
| Albumin: GLP-1 fusion peptide | Human Genome Sciences |
| BIM 51077 | Roche/Ipsen |
| N-terminally truncated GLP-1 derivatives &analogs | Novo Nordisk |
| (lipophilic substituent attached) | PCT/DK99/00081 |
| Derivatives of GLP-1 analogs with a lipophilic substituent | Novo Nordisk |
| | PCT/DK99/00082 |
| | U.S. Pat. No. 6,458,924 |
| N-terminally modified GLP-1 derivatives &analogs with | Novo Nordisk |
| lipophilic substituent attached and protracted profile of | PCT/DK99/00085 |
| action (N-terminal end has a substituent comprising an | |
| optionally substituted 5- or 6-membered ring system) | |
| Derivatives of GLP-1 analogs with a lipophilic substituent | Novo Nordisk |
| (protracted profile of action) | WO 98/08871 |
| GLP-1 fragment as insulinotropic hormone | The General Hospital Corporation |
| | WO 87/06941 |
| GLP-1 derivatives with insulinotropic activity | The General Hospital Corporation |
| | WO 90/11296 |
| GLP-1 analogs exhibiting enhanced stability or an enhanced | Buckley et al. |
| capacity to stimulate insulin production | WO 91/11457 |
| GLP-1 analogs and derivatives (stimulate the secretion or | Eli Lilly &Co. |
| biosynthesis of insulin in poorly functioning beta cells) | EP 0708179-A2 |
| N-terminal truncated GLP-1 and analogs (promote glucose | Eli Lilly &Co. |
| uptake by cells but do not stimulate insulin expression or | EP 0699686-A2 |
| secretion) | |
| GLP-1 analogs or derivatives for increasing the number | Novo Nordisk |
| and/or the size of beta cells and for stimulating beta cell | US 2003/0224983 |
| proliferation | |
| GLP-1 derivatives with a lipophilic substituent and | Novo Nordisk |
| protracted profile of action | U.S. Pat. No. 6,268,343 |
| Pharmaceutical formulations of GLP-1 agonists | Novo Nordisk |
| | US 20030119734 A1 |
| GLP-1 amide, fragment, analogue or derivative | Novo Nordisk |
| | US 20030083259 A1 |
| GLP-1 compositions having protracted action | Novo Nordisk |
| | US 20010006943 A1 |
| GLP-1 &gastrin | Transition Therapeutics |
| | PCT/CA03/ |
| Gastrin formulations | Transition Therapeutics |
| | PCT/CA03/ |
| Derivatives of GLP-1 analogs with a lipophilic substituent | Novo Nordisk |
| (protracted profile of action) | WO 99/43706 |
| GLP-1 and exendin derivatives with just one lipophilic | Novo Nordisk |
| substituent attached to the C-terminal amino acid residue | WO 99/43708 |
| Modified exendins and agonists linked to one or more | Amylin Pharmaceuticals |
| polyethylene glycol polymers | WO 00/66629 |
| Ecarin, a procoagulant protein from Echis carinatus venom | Cohesion Technologies |
| | WO 01/04146 |
| Modified Fragments of GLP-1, exendin 3 and exendin 4 | Conjuchem, Inc. |
| | U.S. Pat. No. 6,514,500 |
| GLP-1 analogs | Novo Nordisk A/S |
| | U.S. Pat. No. 6,451,974 |
| GLP-1 analogs, derivatives and active peptides | Eli Lilly and Company |
| | 6,191,102 |
| GLP-1 Fragments | The General Hospital Corporation |
| | 6,162,907 |
| GLP-1 molecules associated with a divalent metal cation | Eli Lilly and Company |
| | 6,133,235 |
| | 5,977,071 |
| Buccal delivery systems with GLP-1 | Theratech, Inc. |
| | 5,863,555 |
| GLP-1 Analogs | Eli Lilly and Company |
| | 5,981,488 |
| GLP-1 mimics | Bristol-Myers Squibb Company |
| | WO 03/033671 |
| Long lasting GLP-1 | Conjuchem, Inc. |
| | U.S. Pat. No. 6,593,295 |
| | U.S. Pat. No. 6,514,500 |
| | U.S. Pat. No. 6,329,336 |
| Precursor GLP-1 | Genzyme Corporation |
| | WO 03/014318 |
| GLP-1 complexes | Eli Lilly and Company |
| | 6,358,924 |
| Modified peptides | Theratechnologies Inc. |
| | WO 02/10195 |
| GLP-1 and related molecules | Zealand Pharma A/S |
| | WO 2004/005342 |

1. A pharmaceutical composition comprising therapeutically effective amounts of at least one CD3 agonist and at least one gastrin compound that provides beneficial effects relative to each compound alone in the treatment of diabetes, and a pharmaceutically acceptable carrier, excipient, or vehicle.

2.-10. (canceled)

11. A pharmaceutical composition according to claim 1 comprising a synergistically effective amount of a CD3 agonist and a gastrin compound in a pharmaceutically acceptable excipient, carrier, or vehicle.

12. (canceled)

13. A pharmaceutical composition according to claim 1 comprising between about 1000-6000 micrograms CD3 agonist.

14.-17. (canceled)

18. A pharmaceutical composition according to claim 1 wherein the beneficial effect is (a) at least about a 0.05%, 0.1%, 0.5%, 1%, 2%, 5%, 10%, 15%, 20%, 30%, 33%, 35%, 40%, 45%, or 50% increase in pancreatic insulin levels; (b) at least about a 2%, 5%, 10%, 15%, 20%, 30%, 40%, 50%, 60%, 70%, 80%, or 90% decrease in blood glucose levels; and/or (c) a decrease in blood glucose levels for a period of at least about 2 to 4 weeks, 2 to 5 weeks, 3 to 5 weeks, 2 to 6 weeks, 2 to 8 weeks, 2 to 10 weeks, 2 to 12 weeks, 2 to 16 weeks, 2 to 20 weeks, 2 to 24 weeks, 2 weeks to 12 months, 2 weeks to 18 months, or several years following treatment.

19. A pharmaceutical composition according to claim 13 wherein the CD3 agonist is an antibody reactive with CD3 or an F(ab′)₂ fragment of the antibody.

20. A pharmaceutical composition according to claim 19 wherein the CD3 agonist is an anti-CD3 antibody selected from the group consisting of OKT3, hOKT3-γl (Ala-Ala), 145 2Cl 1, YTH 12.5, YTH 12.5.14.2, or CAMPATH-3, or an F(ab′)₂ fragment of the antibody.

21.-22. (canceled)

23. A pharmaceutical composition according to claim 19 wherein the gastrin compound is gastrin 71 [SEQ ID NO. 15], gastrin 52 [SEQ ID NO. 16], gastrin 34 (big gastrin) [SEQ ID NO. 11 or 12], gastrin 17 (little gastrin) [SEQ ID NO. 13 or 14], gastrin 14 [SEQ ID NO. 17], gastrin 8, gastrin 6 [SEQ ID NO.18 or 19], pentagastrin, or tetragastrin.

24. (canceled)

25. A pharmaceutical composition according to claim 20 wherein the gastrin compound is a gastrin 34 or gastrin-17 where there is a methionine or a leucine at position 15.

26.-31. (canceled)

32. A method for treating diabetes in a subject, comprising administering to the subject a combination of a therapeutically effective amount of at least one CD3 agonist and a therapeutically effective amount of at least one gastrin compound to produce a beneficial effect of at least one symptom of diabetes.

33.-35. (canceled)

36. A method as claimed in claim 32 wherein therapeutically effective amounts of the CD3 agonist and the gastrin compound are administered to the subject sequentially.

37. A method according to claim 32 wherein the therapeutically effective amounts of a CD3 agonist and a gastrin compound are synergistically effective amounts.

38.-43. (canceled)

44. A method for treating a subject with diabetes comprising contacting ex vivo a plurality of cells with a a composition of claim 1, optionally culturing the cells, and administering the cells to the subject in need thereof.

45. (canceled)

46. A method according to claim 32 wherein the CD3 agonist is an antibody reactive with CD3 or an F(ab′)₂ fragment of the antibody.

47. A method according to claim 32 wherein the CD3 agonist is an anti-CD3 antibody selected from the group consisting of OKT3, hOKT3γl (Ala-Ala), 145 2Cl 1, YTH 12.5, YTH 12.5.14.2, or CAMPATH-3, or an F(ab′)₂ fragment of the antibody.

48. A method according to claim 32 wherein the CD3 agonist is anti-CD3 mAB hOKT3-γl (Ala-Ala).

49. (canceled)

50. A method according to claim 47 wherein the gastrin compound is gastrin 71 [SEQ ID NO. 15], gastrin 52 [SEQ ID NO. 16], gastrin 34 (big gastrin) [SEQ ID NO. 11 or 12], gastrin 17 (little gastrin) [SEQ ID NO. 13 or 14], gastrin 14 [SEQ ID NO. 17], gastrin 8, gastrin 6 [SEQ ID NO.18 or 19], pentagastrin, or tetragastrin.

51. (canceled)

52. A method according to claim 47 wherein the gastrin compound is a gastrin 34 or gastrin-17 where there is a methionine or a leucine at position 15.

53. A method according to claim 47 further comprising administering a GLP-1 agonist.

54.-56. (canceled)

57. A method for the prevention and intervention of Type 1 diabetes or Latent Autoimmune Diabetes in the Adult (LADA) comprising administering a therapeutically effective amount of a composition as claimed in claim 1.

58. (canceled)

59. A kit form of a composition according to claim 1.


SEQUENCE LISTING (27 sequences)

SEQ ID NO: 1 - protein, 37 aa, Artificial Sequence
  Synthesized peptide
HDEFERHAEGTFTSDVSSYLEGQAAKEFIAWLVKGRG

SEQ ID NO: 2 - protein, 36 aa, Artificial Sequence
  Synthesized Peptide
HDEFERHAEGTFTSDVSSYLEGQAAKEFIAWLVKGR

SEQ ID NO: 3 - protein, 36 aa, Artificial Sequence; identical to SEQ ID NO: 2
  Synthesized Peptide
  MOD_RES (36)..(36): AMIDATION

SEQ ID NO: 4 - protein, 31 aa, Artificial Sequence
  Synthesized Peptide
HAEGTFTSDVSSYLEGQAAKEFIAWLVKGRG

SEQ ID NO: 5 - protein, 30 aa, Artificial Sequence
  Synthesized Peptide
HAEGTFTSDVSSYLEGQAAKEFIAWLVKGR

SEQ ID NO: 6 - protein, 30 aa, Artificial Sequence; identical to SEQ ID NO: 5
  Synthesized Peptide
  MOD_RES (30)..(30): AMIDATION

SEQ ID NO: 7 - protein, 39 aa, Heloderma horridum
  ref <308>: DATABASE ACCESSION NUMBER: P20394
  ref <309>: DATABASE ENTRY DATE: 2004-06-15
  ref <313>: RELEVANT RESIDUES IN SEQ ID NO: (1)..(39)
HSDGTFTSDLSKQMEEEAVRLFIEWLKNGGPSSGAPPPS

SEQ ID NO: 8 - protein, 39 aa, Heloderma suspectum
  ref <308>: DATABASE ACCESSION NUMBER: HWGH4G
  ref <309>: DATABASE ENTRY DATE: 1997-11-21
  ref <313>: RELEVANT RESIDUES IN SEQ ID NO: (1)..(39)
HGEGTFTSDLSKQMEEEAVRLFIEWLKNGGPSSGAPPPS

SEQ ID NO: 9 - protein, 31 aa, Artificial Sequence
  Synthesized Peptide
HGEGTFTSDLSKQMEEAVRLFIEWLKNGGPY

SEQ ID NO: 10 - protein, 31 aa, Artificial Sequence
  Synthesized Peptide
DLSKQMEEEAVRLFIEWLKNGGPSSGAPPPS

SEQ ID NO: 11 - protein, 34 aa, Artificial Sequence
  Synthesized Peptide
  MISC_FEATURE (1)..(1): Xaa is pyroglutamate
XLGPQGPPHLVADPSKKQGPWLEEEEEAYGWMDF

SEQ ID NO: 12 - protein, 34 aa, Artificial Sequence
  Synthesized Peptide
  MISC_FEATURE (1)..(1): Xaa is pyroglutamate
XLGPQGPPHLVADPSKKQGPWLEEEEEAYGWLDF

SEQ ID NO: 13 - protein, 17 aa, Artificial Sequence
  Synthesized Peptide
  MISC_FEATURE (1)..(1): Xaa is pyroglutamate
XGPWLEEEEEAYGWMDF

SEQ ID NO: 14 - protein, 17 aa, Artificial
  Synthesized Peptide
  MISC_FEATURE (1)..(1): Xaa is pyroglutamate
XGPWLEEEEEAYGWLDF

SEQ ID NO: 15 - protein, 101 aa, Artificial
  Synthesized Peptide
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN

SEQ ID NO: 16 - protein, 52 aa, Artificial Sequence
  Synthesized Peptide
DLELPWLEQQGPASHHRRQLGPQGPPHLVADPSKKQGPWLEEEEEAYGWMDF

SEQ ID NO: 17 - protein, 14 aa, Artificial Sequence
  Synthesized Peptide
WLEEEEEAYGWMDF

SEQ ID NO: 18 - protein, 6 aa, Artificial Sequence
  Synthesized Peptide
YGWMDF

SEQ ID NO: 19 - protein, 6 aa, Artificial Sequence
  Synthesized Peptide
YGWLDF

SEQ ID NO: 20 - protein, 31 aa, Artificial Sequence
  Synthesized Peptide
  MISC_FEATURE (31)..(31): Xaa is Pro or Tyr
HGEGTFTSDLSKQMEEEAVRLFIEWLKNGGX

SEQ ID NO: 21 - protein, 40 aa, Artificial Sequence
  Synthesized Peptide
  MISC_FEATURE (2)..(3): Xaa at position 2 is Ser and Xaa at position 3 is Asp or  Xaa at position 2 is Gly and Xaa at position 3 is Phe
HXXGTFITSDLSKQMEEEAVRLFIEWLKNGGPSSGAPPPS

SEQ ID NO: 22 - protein, 44 aa, Artificial Sequence
  Synthesized Peptide
HGEGTFTSDLSKQMEEEAVRLFIEWLKNGGPSSGAPPSKKKKKK

SEQ ID NO: 23 - protein, 6 aa, Artificial Sequence
  Synthesized Peptide
YGWMDF

SEQ ID NO: 24 - protein, 6 aa, Artificial Sequence
  Synthesized Peptide
YGWLDF

SEQ ID NO: 25 - protein, 10 aa, Artificial Sequence
  Synthesized Peptide
GAGAGAGAGA

SEQ ID NO: 26 - protein, 4 aa, Artificial Sequence
  Synthesized Peptide
  MOD_RES (4)..(4): AMIDATION
WMDF

SEQ ID NO: 27 - protein, 4 aa, Artificial Sequence
  Synthesized Peptide
  MOD_RES (4)..(4): AMIDATION
WLDF